(19) United States
(12) Patent Application Publication
(10) Pub. No.: US 2026/0243787 A1
(43) Pub. Date: Aug. 20, 2026

(54) SYSTEMS AND METHODS FOR END-TO-END MATERIAL MANAGEMENT

(71) Applicant: GRAIL, Inc., Menlo Park, CA (US)

(72) Inventors: David Jennions, Raleigh, NC (US); Derek Jenkins, Menlo Park, CA (US)

(73) Assignee: GRAIL, Inc., Menlo Park, CA (US)

(21) Appl. No.: 19/413,398

(22) Filed: Dec. 9, 2025

Related U.S. Application Data
(60) Provisional application No. 63/729,697, filed on Dec. 9, 2024.
(60) Provisional application No. 63/729,719, filed on Dec. 9, 2024.
(60) Provisional application No. 63/729,745, filed on Dec. 9, 2024.

(51) Int. Cl. G01N 35/00

(52) U.S. Cl. CPC G01N 35/00732; G01N 35/0092; G01N 2035/00801

(57) ABSTRACT

Aspects of the disclosure may be directed to an automated sample processing system having one or more processors and one or more computer readable media storing instructions executable by the one or more processors to perform one or more functions. The one or more functions may include identifying that a sample in a laboratory workflow controlled by the system is in a buffer work cell, receiving operational context data associated with the laboratory workflow indicating an event affecting a subsequent processing step for the sample in the laboratory workflow, and dynamically adjusting a dwell time for the sample in the buffer work cell based on the received operational context data.

### CROSS-REFERENCE TO RELATED APPLICATIONS

This application claims the benefit of priority to U.S. Provisional Patent Application No. 63/729,697, filed on Dec. 9, 2024, U.S. Provisional Patent Application No. 63/729,719, filed on Dec. 9, 2024, and U.S. Provisional Patent Application No. 63/729,745, filed on Dec. 9, 2024, all of which are incorporated herein by reference in their entireties.

### TECHNICAL FIELD

The present disclosure relates generally to computer systems designed for the management of workflows across various subsystems and, more specifically, to systems and methods for autonomously processing samples in analytical assays and dynamically managing the inventory associated therewith.

### BACKGROUND

The processing of samples, such as biological samples, in analytical assays may involve multiple complex steps that traditionally require significant manual intervention. Existing systems often suffer from inefficiencies due to the lack of integration between sample processing, inventory management, and reprocessing capabilities. This fragmentation leads to increased downtime, errors, and bottlenecks with assay workflows. Additionally, the traditional reliance on human involvement introduces variability and potential errors, thereby compromising the efficiency and accuracy of the overall process.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, systems and methods are described for dynamically and autonomously managing the end-to-end workflow of sample processing, such as biological sample processing.

Aspects of the disclosure may be directed to an automated sample processing system having one or more processors and one or more computer readable media storing instructions executable by the one or more processors to perform one or more functions. The one or more functions may include identifying that a sample in a laboratory workflow controlled by the system is in a buffer work cell, receiving operational context data associated with the laboratory workflow indicating an event affecting a subsequent processing step for the sample in the laboratory workflow, and dynamically adjusting a dwell time for the sample in the buffer work cell based on the received operational context data.

Aspects of the disclosure may also be drawn to an automated sample processing system having one or more processors and one or more computer readable media storing instructions executable by the one or more processors to perform one or more functions. The one or more functions may include receiving an indication that an amount of a material at a local storage compartment associated with a laboratory workflow has fallen below a predetermined threshold; determining a status of one or more samples being processed in accordance with the laboratory workflow and, depending on the status, a projected movement of the one or more samples on a conveyance platform of the system; and initiating a replenishment protocol responsive to receiving the indication to increase the amount of the material above the predetermined threshold and based on the projected movement of the one or more samples on the conveyance platform.

Further aspects of the disclosure may be drawn to an automated sample processing system having one or more processors and one or more computer readable media storing instructions executable by the one or more processors to perform one or more functions. The one or more functions may include receiving an indication of a shutdown event for a laboratory workflow controlled by the system; implementing a shutdown procedure to address the shutdown event, wherein the shutdown procedure causes one or more samples involved in the laboratory workflow to continue processing to a predefined safe stopping point; and halting at least a subset of the operations of the system at a conclusion of the shutdown procedure.

Still other aspects of the disclosure may be drawn to an automated sample processing system having one or more processors and one or more computer readable media storing instructions executable by the one or more processors to perform one or more functions. The one or more functions may include identifying a position of a sample in a laboratory workflow controlled by the system; determining that the position of the sample in the laboratory workflow corresponds to an automatic storage position; causing a portion of the sample to be aliquoted to create a matched aliquoted sample; and storing, responsive to the determining, the matched aliquoted sample in a storage location.

Additional aspects of the disclosure may be drawn to an automated sample processing system having one or more processors and one or more computer readable media storing instructions executable by the one or more processors to perform one or more functions. The one or more functions may include identifying a position of a sample in a laboratory workflow controlled by the system; identifying a status of an activity work cell in a laboratory workflow that is downstream of the position of the sample in the laboratory workflow; determining that the status of the activity work cell with respect to the position of the sample requires placement of the sample in a buffer work cell; and transporting the sample to the buffer work cell.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate several embodiments, and together with the description, serve to explain the principles of the disclosure.

FIG. 1 depicts a block diagram illustrating a hierarchical software architecture for executing the methods described herein, according to one or more embodiments of the present disclosure.

FIG. 2 depicts a diagram illustrating the input and output functions of a top, or first, software layer in the hierarchical software architecture, according to one or more embodiments of the present disclosure.

FIG. 3 depicts a diagram illustrating the input and output functions of a second software layer in the hierarchical software architecture, according to one or more embodiments of the present disclosure.

FIG. 4 depicts a diagram illustrating the input and output functions of a third software layer in the hierarchical software architecture, according to one or more embodiments of the present disclosure.

FIG. 5 depicts a diagram illustrating an exemplary integration of activity work cells, buffer work cells, and shared storage locations, according to one or more embodiments of the present disclosure.

FIG. 6 depicts a diagram designating the position of repeat points in a multi-stage process for the automated handling and processing of biological samples, according to one or more embodiments of the present disclosure.

FIG. 7 depicts an exemplary flow diagram that delineates a decision-making process associated with automatically storing and retrieving samples for reprocessing.

FIG. 8 depicts a diagram designating the position and type of safe storage points in a multi-stage process for the automated handling and processing of biological samples, according to one or more embodiments of the present disclosure.

FIG. 9 depicts an exemplary flow diagram that delineates a decision-making process associated with automatically storing and retrieving samples for reprocessing.

FIG. 10 depicts an example computing system, according to one or more embodiments of the present disclosure.

### DETAILED DESCRIPTION OF EMBODIMENTS

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

The processing of biological samples for analytical assays (e.g., such as DNA extraction, plasma isolation, sequencing preparation, etc.) is inherently complex and requires precise handling, timing, and coordination across multiple steps. Traditionally, these workflows have relied heavily on manual intervention, which introduces inefficiencies, increases the likelihood of errors, and results in significant downtime. More particularly, the manual nature of these processes not only slows down the workflow but also makes it difficult to manage the simultaneous movement and processing of samples and materials without causing bottlenecks.

Moreover, tracking and managing the inventory of reagents, consumables, and other materials necessary for these processes is challenging, often leading to interruptions in the workflow when supplies run low or are unavailable at critical times.

Conventional attempts to address these challenges have focused on automating individual steps within the workflow, such as using robotic pipetting systems or automated DNA extraction machines. Additionally, separate inventory management systems have been developed to track and replenish materials independently from the sample processing workflow. In some cases, dedicated transport systems for samples and inventory items have been implemented to reduce conflicts and improve efficiency. However, these approaches have significant limitations. Automating discrete steps without a unified system may lead to inefficiencies, as each step may still wait for others to complete, resulting in increased downtime. Additionally, separate inventory systems may be prone to synchronization issues, causing delays when materials are not available when needed, and these systems may struggle to maintain an accessible audit trail for tracking reagent quality and handling errors. Furthermore, manual interventions for error recovery may be time-consuming and prone to human error, leading to wastage of samples and reagents, or even sample contamination. Lastly, dedicated transport systems, while useful, may still cause bottlenecks during high-volume operations due to the lack of dynamic prioritization and resource allocation.

Additionally to the foregoing, laboratory workflows, especially those involved in the processing and analysis of biological samples, require high precision and efficiency. Conventional laboratory setups, while often labeled as autonomous, are typically only semi-autonomous. More particularly, critical steps such as sample preparation, transportation between machines, initiating specific processes, and attending to system errors still depend heavily on human intervention. This dependency introduces variability and potential errors, adversely impacting the efficiency and accuracy of the workflows. Moreover, these systems are difficult to scale without a proportionate increase in workforce and space requirements, making it challenging to meet higher throughput demands. There is a need for a truly autonomous system that can handle the variability and complexity of laboratory workflows from start to finish with little, if any, human involvement.

Conventional efforts to automate laboratory workflows have primarily focused on either automating discrete steps of the overall process or fully centralizing control within a single system. With respect to the former, step-specific automation may involve automating individual steps of the laboratory workflow rather than the entire process. More particularly, certain machines in the overall workflow may be configured to perform specific tasks, such as sample preparation, analysis, or data collection. Examples of step-specific automation may include liquid handling robots that automate pipetting and reagent dispensing, automated PCR systems that perform polymerase chain reactions with minimal human intervention, and robotic sample storage systems that automatically store and retrieve samples from refrigerated units.

However, the advantages provided by these step-specific automation processes do not address the need for a seamless, end-to-end workflow. More particularly, human operators are still required to transport samples between machines, initiate processes, and handle exceptions. This type of fragmented automation introduces inconsistencies and potential errors, compromising the overall efficiency and accuracy of the workflow. Additionally, the throughput of step-specific automation systems is constrained by the need for manual handling between steps, and scaling these systems requires proportionally increasing the number of human operators. Furthermore, maintenance and downtime are additional issues, as individual machines require regular maintenance and calibration, leading to disruptions in the workflow and reduced productivity or redundancy of said individual machines can be added at great additional cost and physical space.

With respect to the latter of the two conventional solutions, centralized automation systems are designed to manage all aspects of the laboratory workflow through a single, overarching controller. These systems integrate various automated machines and instruments to perform specific tasks within the workflow. Some components of centralized systems may include robotics arms for moving samples between different workstations or components of workstations, automated liquid handling systems for tasks such as pipetting and reagent addition, and integrated software platforms that provide a unified interface for controlling all automated components.

Despite their promise, centralized automation systems face significant issues. For instance, one of the primary problems is computational overload. More particularly, centralized systems require a single controller to manage numerous variables, including machine status, sample location, and timing for each process step, to name a few. Attempts to harmoniously manage these variables has conventionally led to computational overload, thereby causing delays, inefficiencies, and frequent system failures. Additionally, these systems still depend heavily on human operators to prepare samples, initiate processes, and troubleshoot issues. This reliance on human intervention introduces variability and potential errors, reducing overall efficiency and accuracy.

Scalability is another challenge encountered by centralized systems. Specifically, scaling these systems to handle higher throughput (e.g., population-level sample analysis) involves adding more automated machines, which increases the use of and reliance on the central controller, and results in computational overload. This scaling approach is limited by the controller's ability to manage additional components and the increased space required for additional machinery. Integration and compatibility issues also arise when integrating automated components from different vendors, making seamless communication and coordination between diverse machines and instruments complex and prone to errors. Lastly, centralized systems typically require complete shutdowns to conduct maintenance, upgrades, or repairs, leading to significant workflow disruptions and productivity loss.

Additionally to the foregoing, some industries, such as the automotive sector, have successfully implemented end-to-end automation, benefitting from the standardized and repetitive nature of their processes. In these industries, automation systems are designed to handle a relatively narrow range of tasks with consistent inputs and outputs, allowing for efficient and predictable operations. In contrast, laboratory workflows present a unique challenge due to their inherent variability. More particularly, laboratories often deal with a wide range of sample characteristics, sample quantities, sample types, processing methods, and analytical requirements, making it difficult to standardize workflows in the same way as the manufacturing industries. This variability introduces complexity in automation, as systems must be flexible enough to adapt to different protocols and procedures or respond to variations in a given sample including its reactions to the components of an assay. As a result, fully automating laboratory workflows requires advanced systems capable of dynamic decision-making and adaptability, which are often more challenging to develop and implement than the relatively straightforward automation systems used in industries with less variability.

Accordingly, the novel concepts described herein are generally directed to an integrated end-to-end material management system for autonomous workflow management in biological sample processing. The system contains several innovative aspects, including buffer work cells that streamline workflow transitions and workflow distribution and avoid downtime, autonomous delivery of resources to local storage compartments accessible to activity work cells, and the ability to store excess sample derivatives for potential reprocessing in the event of deviations or errors. The system contains a centralized conveyance platform that may be leveraged to dynamically prioritize and coordinate the movement of samples and inventory, optimizing resource allocation and avoiding bottlenecks.

By integrating buffer work cells, local storage, and centralized conveyance platforms, the system promotes seamless transitions and continuous processing, which may avoid downtime and inefficiencies associated with conventional systems. The system may contain real-time inventory monitoring and autonomous replenishment capabilities so that materials are available when needed, reducing disruptions and delays. Furthermore, the system may be configured to store and reprocess excess sample derivatives, which may promote quick recovery from errors or unexpected results without the need for new samples, thereby reducing wastage and enhancing workflow reliability while maintaining turnaround time for the overall process. Additionally, the system may be able to dynamically adjust dwell times based on real-time data, which may further improve overall efficiency and workflow continuity.

Additionally to the foregoing, the novel computer system is designed to achieve complete autonomy in laboratory workflows by leveraging a hierarchical software architecture, in which each layer of software in the hierarchical architecture is purposefully left generally ignorant or agnostic of the other layers. This novel system includes multiple independent software layers that distribute computational tasks at different layers of abstraction, thereby reducing data complexity and preventing computational overload. At the highest level, the top software layer orchestrates the overall workflow, managing the flow of samples and materials between various work cells based on context data such as assay protocols, system component status, and inventory levels. The top software layer operates without awareness of the details of operations in the various work cells. The second software layer, functioning as a scheduler, manages tasks within individual work cells by mapping required work to available resources, allowing for the simultaneous processing of multiple sample batches. The second software layer operates without awareness of the other work cells or the broader workflow context. The third software layer controls specific instruments within the work cells, executing discrete operations without awareness of the other instruments in the work cell or the broader workflow context.

The innovative architecture described above ensures that each software layer operates independently and agnostically, enhancing the system's reliability and scalability. Additionally, the system's modular design allows for the addition or removal of hardware components, such as work cells and instruments, without necessitating system-wide shutdowns, thus maximizing uptime and operational efficiency. Moreover, the novel system may include an integrated information repository, which facilitates communication between layers, storing progress and quality assurance data and enabling the top software layer to monitor the overall health and workflow status of the system. Accordingly, by eliminating the need for human intervention in work allocation, sample handling, and movement, this fully autonomous system reduces the risk of human error, thereby enhancing efficiency and enabling the processing of large numbers of samples (e.g., millions of samples) with precision and speed.

A system that incorporates the innovative concepts described herein may be well suited, for example, for sample preparation and analysis in cancer detection analysis, where precision and consistency are important considerations. For instance, the system may be capable of efficiently processing a large quantity of samples or different types of biological samples, such as liquid (e.g., blood, urine, saliva, stool, cerebrospinal fluid, pleural fluid, interstitial fluid, etc.), tissue, or biopsy specimens, to extract nucleic acids and other biomarkers important for cancer diagnosis. The system's hierarchical software architecture may be configured to manage these processes, which may not only increase the efficiency of the sample preparation process but also reduce the potential for human error, thereby enhancing the reliability and accuracy of downstream cancer detection assays, while maintaining or even improving long-term costs.

The concepts described herein introduce several significant technical improvements. For instance, by dividing tasks among multiple independent software layers and purposefully keeping the software layers agnostic to one another, the system reduces the computational burden on any single layer. Each layer is responsible for specific functions, allowing for more efficient processing and avoiding the risk of system overload. This distribution enhances the system's reliability and performance, preventing the computational bottlenecks that commonly plague centralized systems.

Additionally, the system's architecture supports modifications, repairs, and updates while remaining operational. This “online” maintenance capability promotes continuous operation and minimal disruption, a significant improvement over conventional systems that require complete shutdowns for even routine maintenance tasks. In an aspect, dynamic task reallocation allows the top software layer to dynamically reassign work to alternative work cells or instruments if a specific component needs to be temporarily taken offline for servicing. Furthermore, the modular design of the novel system allows it to easily scale by the addition or removal of work cells and instruments as needed, without significantly compromising performance. This scalability is achieved without the need for extensive reconfiguration or system downtime, making it possible to adapt to varying throughput requirements and evolving laboratory needs. The ability to integrate additional hardware components ensures that the system can grow in response to increasing demand or new technological developments or shrink in response to decreased throughput needs or upstream efficiencies. Furthermore, the system is configured to autonomously manage complex workflows, including the movement and processing of samples, with precision and speed. This autonomy enables the system to process millions of samples accurately, improving throughput and reliability.

Furthermore, the system may enhance resource-management through real-time monitoring and autonomous replenishment of materials, a capability made possible by its integrated inventory management system and local storage solutions. More particularly, by continuously or periodically tracking reagent levels, instrument status, and sample location within the system, the system dynamically allocates resources as they are needed, promoting a seamless workflow continuity and reducing downtime. This may not only optimize the use of laboratory space and equipment, but it may also improve throughput, making the system scalable and adaptable to varying workloads. Additionally, the ability to dynamically adjust dwell times based on real-time data may further refine the efficiency of the workflow, ensuring that samples are processed quickly and accurately without unnecessary delays.

Another improvement to the technical field is represented by the system's sophisticated error-handling and crash recovery protocols. Unlike conventional systems that may halt abruptly during unexpected shutdowns, the described system may incorporate a unique crash recovery mechanism that allows samples to continue processing until they reach predefined safe points, where they can be stored. This promotes the maintenance of sample integrity even during unforeseen events, such as power outages or other critical failures, IT failures, contamination events, and promotes a smooth resumption of operations upon system restart. By inhibiting the risk of data loss and sample damage, the system provides a more reliable and resilient solution for high-throughput environments.

Additionally, the system represents an advancement in assaying technology, specifically enhancing high-throughput capabilities. More particularly, traditional assaying workflows often suffer from inefficiencies and limitations due to the need for manual interventions, discrete automation of individual steps, and lack of integration between different stages of the process. By contrast, the system described herein integrates all stages of biological sample processing into a cohesive, fully automated workflow that minimizes human involvement and optimizes resource allocation. By integrating multiple work cells, equipped with specialized instruments and local storage, the system is enabled to handle various assay tasks simultaneously (e.g., from sample preparation and reagent addition to analysis and data collection), without manual handoffs or coordination.

The subject matter of the present disclosure will now be described more fully hereinafter with reference to the accompanying drawings, which form a part hereof, and which show, by way of illustration, specific exemplary embodiments. An embodiment or implementation described herein as “exemplary” is not to be construed as preferred or advantageous, for example, over other embodiments or implementations; rather, it is intended to reflect or indicate that the embodiment(s) is/are “example” embodiment(s). Subject matter may be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware, or any combination thereof. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” or “in some embodiments,” or “in one aspect” or “in some aspects” as used herein does not necessarily refer to the same embodiment or aspect, and the phrase “in another embodiment” or “in another aspect” as used herein does not necessarily refer to a different embodiment or aspect. It is intended, for example, that claimed subject matter include combinations of exemplary embodiments in whole or in part.

Although the present disclosure is primarily described in the context of biological sample processing, it should be understood that the system architecture is not limited to this application and may be adapted for a wide variety of other fields and tasks that require precise, multi-step processing. For instance, this system may be effectively employed in the manufacturing industry for the assembly of electronic components, where different work cells may be configured to handle tasks such as soldering, component placement, and quality control. Similarly, the concepts described herein may be utilized in the pharmaceutical industry for drug formulation and packaging, ensuring that each step, from ingredient mixing to final packaging, is carried out accurately and efficiently without human intervention.

The computer system described herein may be designed to handle a wide variety of sample types, making it suitable for diverse applications across multiple industries. The system's modular and flexible architecture allows it to efficiently process samples with different properties and requirements. For instance, non-limiting exemplary types of samples that may be processed through the disclosed system include: biological samples (e.g., tissue samples, blood samples, urine samples, saliva samples, fecal samples, or other fluid samples, etc.), environmental samples (e.g., water samples, soil samples, etc.), food and beverage samples, forensic samples (e.g., crime scene samples, toxicology samples, etc.), industrial samples (e.g., pharmaceutical samples, chemical samples, etc.), and the like. In an aspect, the versatility of the system may allow it to be adapted for specific sample types and processing requirements. Work cells within the system may be configured with specialized equipment and reagents tailored to the unique properties of each sample type, ensuring accurate and efficient processing from start to finish. This capability makes the system a valuable tool across various fields, enhancing the reliability and throughput of analytical workflows.

### Definitions

The following definitions clarify key terms used throughout this disclosure to describe the various aspects of the autonomous workflow management system.

As used herein, a “hierarchical software architecture” may refer to a layered structure of the software system, where each layer operates independently and performs specific functions within the overall workflow. For instance, this architecture may include a top software layer, a second software layer, and a third software layer, each managing different aspects of the processing workflow. Although three software layers are used in descriptions and many examples herein, more or fewer layers may be used in certain embodiments based on the complexity of the software system and individual tasks thereof.

A “workflow” may refer to the high-level ordered set of work necessary to complete an overall goal. It encompasses the sequence of stages that need to be executed to achieve the desired end result. In the context of this disclosure, a workflow dictates the progression of sample processing from one stage to the next, outlining the major phases required to process and analyze biological samples. For instance, given a DNA extraction and analysis scenario, the workflow may designate the following sequence of stages: sample collection, sample preparation, DNA extraction, DNA purification, DNA quantification, sequencing preparation, sequencing, and data analysis.

A “recipe” may refer to the set of parameters that define how a workflow should actually be executed. These parameters may include specific instructions, conditions, and/or settings that tailor the execution of a workflow to meet the particular requirements of different products or samples. Recipes may vary significantly between different types of samples processed, products, assays, or desired outputs, providing the detailed operational guidelines that direct how each step in the workflow is carried out. In essence, the recipe adds the specificity and customization to the more generalized framework of a workflow. For instance, given the workflow for a DNA extraction and analysis scenario, as previously described above, the recipe for executing the workflow for a set of blood samples may include: preparation of the samples by centrifuging the blood at 1500 g for 10 minutes to separate plasma and utilize a specific lysis buffer for lysing cells, using a specified enzyme or chemical reaction to prepare the DNA for epigenomic analysis, extracting DNA utilizing a silica-based extraction kit and incubating the samples with proteinase K at a specific temperature for a specified time, purifying the DNA using an ethanol wash and eluting the DNA in a specified volume of elution buffer, performing DNA quantification using a spectrophotometer to measure DNA concentration, performing sequencing preparation via utilizing a specific library preparation kit for the sample type and performing PCR amplification (e.g., with a specified number of cycles), sequencing the samples using a predefined sequencing platform, and aligning sequences, performing variant calling, or other analyses using a specific software, model, or analytical pipeline. Should DNA need to be extracted from urine, saliva, or fecal samples instead of blood samples, a different recipe may be employed (e.g., different types of collection tubes, different centrifugation parameters, etc.) up to a certain point (e.g., once DNA is extracted). Although the specific example above is provided as an example of a recipe, it is understood that other recipes, or modifications to this example recipe, may be used. Embodiments of the specification are not limited to the particular recipe set forth above.

A “workflow process” combines the workflow and the recipe. More particularly, the workflow process may correspond to a predefined, structured set of instructions and procedures designed to guide the sequential processing of a sample through various stages of analysis or production. In the context of this application, the workflow process serves as a comprehensive roadmap that outlines each specific step required to process a sample from start to finish, ensuring that all necessary tasks are performed in the correct order, by the correct work cell, and under the appropriate conditions. In some aspects, the workflow process may not specify the exact software layers and/or instruments that may be used to process the sample. For example, if a work cell includes three centrifuges A, B, and C, the workflow process may simply indicate that one of the steps that is used to process the sample is centrifugation, and may not specify which particular centrifuge to use. In another example, if a work cell includes three centrifuges A, B, and C, the workflow process may simply indicate that the sample may be processed by only centrifuge A or B, because centrifuge C may be down or temporarily occupied or otherwise unavailable. A second software layer associated with a work cell may then use the information in the workflow process to decide which of centrifuges A and B is the best choice to process the sample, e.g., in order to improve the efficiency of sample processing.

A “Laboratory Information Management System (LIMS)” may refer to a data storage repository that records information related to sample processing. LIMS may be configured to store details such as sample status, workflow progress, and system help. Each software layer may publish execution data to LIMS, and the top software layer may periodically or continuously access this repository to make informed decisions and/or to modify the recipe and/or workflow process (e.g., for a given biological sample in processing).

A “work cell” may refer to a cluster of instruments, each designed to perform specific steps in the process. Work cells may operate semi-independently, contributing to the system's modularity and scalability. Different work cells may be configured for various stages, e.g., including DNA extraction, amplification, or sequencing preparation.

An “activity work cell” may correspond to a specialized unit within an autonomous workflow management system designed to execute specific workflow steps. Each activity work cell may contain a group of equipment and instruments necessary for processing steps according to predefined workflows. For instance, the equipment within an activity work cell may include one or more robotic components, centrifuges, pipetting instruments, PCR machines, etc. Each type of activity work cell may be primarily defined by the equipment involved, the input expected (e.g., sample in a specific state and/or reagents), and the anticipated output.

A “buffer work cell” may correspond to a specialized storage unit within the autonomous workflow management system that is designed to store materials, reagents, and intermediate products for on-demand usage by activity work cells. Buffer work cells do not execute workflow steps but facilitate smooth transitions and continuous operations by ensuring that materials are readily available. Buffer work cells may include equipment such as refrigerators, freezers, climate control systems (e.g., humidifiers, dehumidifiers), ambient storage units, and automated retrieval systems (e.g., robotic arms, conveyance systems, etc.). In some aspects, buffer work cells may also include multi-temperature storage compartments to maintain the stability and integrity of stored materials, ranging from ultra-low temperatures to ambient conditions.

A “conveyance platform” may refer to one or more mechanisms that connect work cells and facilitate the movement of samples between them. The conveyance platform may ensure the transfer of samples and assay materials (e.g., reagents and disposables), allowing the system to maintain continuous operation. Exemplary conveyance platforms may include one or more hardware components, including pucks, tracks, belts, grippers, etc. A conveyance platform may be centralized (e.g., comprise one or more large-scale installations connecting many work cells along a defined path) or decentralized (e.g., comprise many components capable of independent movement, such as an autonomous transport device capable of connecting two end points without a defined path or order). In some instances, a conveyance platform may include centralized or decentralized components.

A “robotic component” may be a component in the work cell that is designed to perform a variety of precise and repetitive tasks that are important for processing samples. As used herein, the robotic component may correspond to, e.g., a “robotic arm.” The robotic arm is a highly versatile, mechanical device that may be equipped with multiple joints and degrees of freedom, which may mimic the dexterity and range of motion of a human arm, enabling it to execute complex maneuvers with high precision and accuracy. This may include tasks such as pipetting exact volumes of liquid, transferring samples between different containers or instruments, and/or from the conveyance platform to different containers or instruments, mixing reagents, and positioning samples within various instruments. In the context of this application, a robotic component may refer to a single robotic component or a series of robotic components.

For the purposes of this disclosure, the terms “stage,” “step,” “task,” and “operation” have specific definitions and are not synonymous.

A “stage” may refer to a broader portion of the assay workflow that encompasses a series of related processes aimed at achieving a specific intermediate goal. Stages represent major phases within the overall workflow, such as DNA pre-extraction, extraction, and enrichment. For example, the pre-extraction stage may involve initial sample preparation and handling, the extraction stage may involve isolating target molecules, and the enrichment stage may involve enhancing the concentration and purity of the extracted molecules. The top layer of software may coordinate which stage a sample moves to next. In some instances, a single stage may be split between two work cells to optimize resource utilization and enhance processing efficiency. This division may, if utilized, allow the system to balance workloads by distributing actions based on the availability and capability of each work cell.

A “step” may refer to a discrete process within a stage. Steps are specific actions that need to be performed to progress through a stage, each with defined inputs and outputs. The second software layer may coordinate these steps, mapping required tasks to available resources within a work cell. For example, within the extraction stage, steps may include digestion, extraction consolidation, and washing and elution.

A “task” may correspond to a specific unit of work within a step, often corresponding to a single function performed by an instrument or set of closely related functions. For instance, to execute the step of digestion, an instrument may perform a series of tasks including centrifugation, peeling, internal storage, plasma transfer, mixing, heating, incubation, etc. Each of these tasks is a distinct unit of work that can be assigned to a specific instrument or piece of equipment designed to perform that function.

An “operation” may correspond to the most granular level of activity, representing a logical, distinct part of a task, typically involving a single motor control or a specific action performed by the instrument. More particularly, operations may be thought of as the basic actions that, when combined, accomplish a task. For instance, in the task of adding a reagent to a sample tube, there may be an operation to draw the reagent (e.g., a motor is controlled to raise the plunger, creating a vacuum that draws the reagent up into the pipette tip) and another operation to dispense the reagent (e.g., the motor may be controlled to lower the plunger, pushing the reagent out of the pipette tip and into the sample tube).

A “sample” may refer to an individual sample or a batch of samples, for convenience, unless specified otherwise. Samples may be stored in sample tubes (e.g., individually or pooled) or may be stored in devices configured to hold and separate multiple samples (e.g., multi-tube racks, wells of multiwell plates) to enable simultaneous manipulation and treatment of multiple samples.

A “memory device” may be any suitable device that can store electronic data. A suitable memory device may contain a computer readable medium that stores instructions that can be executed by a processor to implement a desired method. Examples of memory devices may contain one or more memory chips, disk drives, etc. Such memory devices may operate using any suitable electrical, optical, and/or magnetic mode of operation.

A “processor” may refer to any suitable data computation device or devices. A processor may include one or more microprocessors working together to accomplish a desired function.

### System Software Architecture

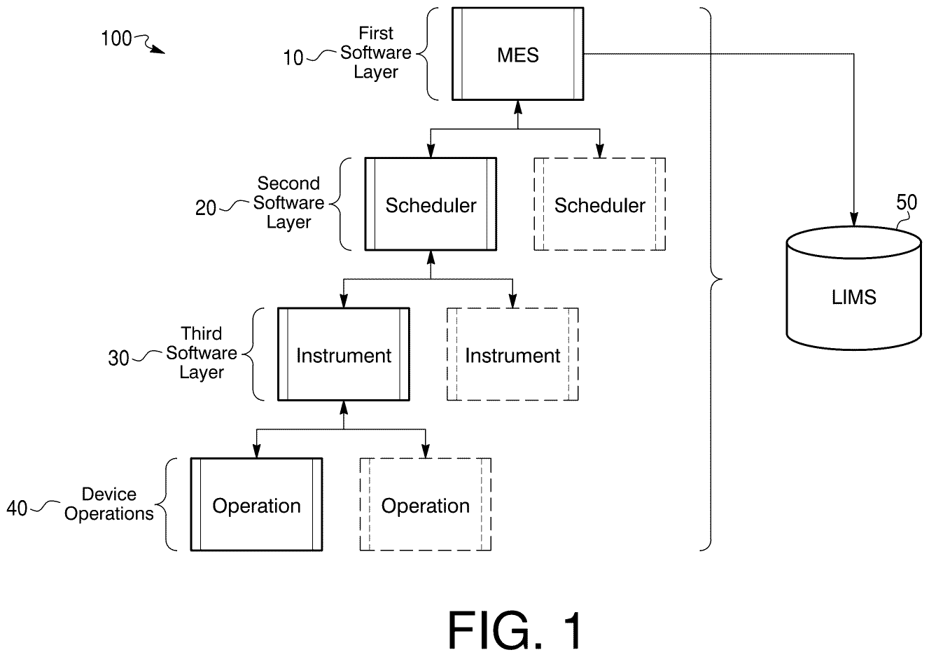

FIG. 1 depicts a block diagram illustrating a hierarchical software architecture 100 for a fully automated system, according to embodiments of the disclosure. The software architecture 100 may contain a plurality, e.g., three, software layers, including the top or first software layer 10, the second software layer 20, and the third software layer 30, with each layer responsible for its own specific set of data. The third software layer 30, which corresponds to instrument control within a work cell, may be responsible for executing specific device operations 40, as further discussed herein. In an aspect, the three software layers 10, 20, 30 may be in the form of software components that are stored on or in a memory and/or computer readable medium and that work with one or more processors (e.g., data processors) residing on one or more computer apparatuses. For example, all three layers 10, 20, 30 may reside on or in a computer readable medium on one computational apparatus with one or more processors (e.g., microprocessors). Alternatively, the three layers 10, 20, 30 may reside on or in three computer readable media residing on three separate computational apparatuses, each with one or more processors (e.g., microprocessors). In some aspects, the top software layer 10 may reside in a first computational apparatus (e.g., a first server computer, virtual machine, or thread), the second software layer 20 may reside in a second computational apparatus (e.g., a second server computer, virtual machine, or thread), and the third software layer 30 may reside in a third computational apparatus (e.g., a third server computer, virtual machine, or thread). Alternatively, a subset of the three layers 10, 20, 30 may reside on or in two computer readable media residing on two separate computational apparatuses, each with one or more processors (e.g., microprocessors), such that two software layers reside on or in one computer readable media and one software layer resides on its own computer readable media.

The Laboratory Information Management System (LIMS) 50 is a component within the hierarchical software architecture of system 100. Serving as a centralized information repository, LIMS 50 stores data generated throughout the workflow process. More particularly, LIMS 50 functions to provide a comprehensive database where relevant information about samples, processes, and system status is recorded. This includes data on sample identity, processing steps and results, reagent usage, and the status of various system components. By maintaining a centralized repository of such information, LIMS 50 enables the system to track the progress of each sample in real time, ensuring that all actions are accurately documented and traceable. This traceability promotes the integrity of the workflow and compliance with regulatory standards. In an aspect, LIMS 50 interfaces with each of the three software layers 10, 20, 30, facilitating the flow of information across the system 100. One or more layers of software 10, 20, 30 may publish information to LIMS 50 while interfacing with samples or while awaiting samples. As further discussed herein, the top software layer 10 may access LIMS 50 to obtain context data, such as inventory levels, sample positions in the workflow process, instrument and component health indications, and the like, which are necessary for high-level decision-making and workflow orchestration. It is important to note that although FIG. 1 and the balance of this disclosure discusses a software architecture that contains three software layers, such a designation is not limiting. Specifically, fewer or additional software layers may be utilized in a system to facilitate the concepts described herein. For instance, instruments within the system may also be configured to oversee specific sub processes independently. This capability may allow individual instruments to manage tasks such as calibration, quality control checks, and data validation.

The fully automated system for which the hierarchical software architecture 100 is constructed may include a variety of different hardware components. Provided below are descriptions of hardware components that may be utilized in the fully automated system. However, it is important to note that the number and type of hardware components may vary between systems and may be tailored to the overall goal of the fully automated system. Additional detail regarding how these hardware components interact and/or are controlled by the various system layers 10, 20, 30 are further provided herein.

The fully automated system may include one or more work cells which, in general, refer to a cluster of instruments that are each designed to execute various tasks in the furtherance of specific steps in the process workflow. More particularly, each work cell may be equipped with a variety of instruments and components tailored to the specific sample processing step(s) it was organized to perform. Some or all work cells may include a robotic component, such as a robotic arm, that may be linked to and controlled by a second software layer responsible for coordinating and executing tasks within the work cell. For instance, as an example, a pre-amplification processing work cell may be designed and equipped to handle various preparatory actions required for biological sample processing. It may contain a robotic arm capable of performing actions such as pipetting, mixing, and/or transferring samples between different instruments. This robotic arm may ensure that each step, task, and/or operation is repeatedly executed accurately, reducing the risk of human error. In an aspect, the work cell may additionally be equipped with other instruments, such as an automated pipettor (e.g., that facilitates precise liquid handling, allowing for the accurate addition of reagents or samples into plates or tubes), a centrifuge (e.g., which is utilized to separate components of the sample based on density), a heater/shaker (e.g., which may provide the necessary heating and agitation to ensure proper mixing and reaction conditions during various processes), an ID scanner, such as a one-dimensional or two-dimensional barcode scanner, (e.g., which may be utilized to ensure accurate identification and data logging), and the like. As another example, another type of work cell may be a post-amplification processing work cell, which may be designed to handle tasks related to the amplification and subsequent processing of biological samples. Such a work cell may include a variety of equipment, some or all of which may be different from the pre-amplification work cell, including: a polymerase chain reaction (PCR) machine (e.g., which is utilized for amplifying DNA samples through PCR, thereby increasing the quantity of DNA), a thermocycler (e.g., which may be utilized to facilitate the cycling of temperatures necessary for the denaturation, annealing, and extension phases of PCR), an automated gel electrophoresis system (e.g., which may be utilized for separating and analyzing DNA fragments, providing a means to verify the success and quality of the amplification process), and/or other devices not explicitly listed here.

Additionally to the foregoing, buffer work cells may be included in the system and may serve as intermediary storage units that help regulate the flow of samples and materials between different stages of processing. More particularly, unlike the activity work cells described above, these specialized units may not perform active processing steps or tasks. Instead, they may be configured to act as temporary holding areas for samples, reagents, and other consumables (e.g., items that may be used up or consumed during laboratory processes, such as reagents and chemicals, plastics and glassware, filters, membranes, laboratory gases, etc.). By providing a buffer between active work cells and the conveyance platform, these buffer work cells help to smooth out the fluctuations in processing rates, ensure smooth transition from the conveyance platform to an activity work cell, and manage unexpected delays, thereby allowing subsequent stages of the workflow process to proceed without interruption. In an aspect, buffer work cells may include one or more storage environments, such as one or more of ambient, refrigerated, and/or freezer storage environments, to maintain the integrity of samples and reagents. Additionally, in some aspects, they may enable the system to balance workloads by temporarily storing batches of samples when downstream work cells are occupied or during peak processing times, or may allow consumables to be delivered to work cells at times when samples are not being actively conveyed along a route to reduce sample down time and avoid unnecessary traffic on the conveyance platform. In particular, because a robotic arm may be required to place reagents, consumables, and other items, the addition of a buffer work cell with accompanying robotic arm upstream of an activity work cell ensures that the activity work cell is not slowed down by actions involving the repositioning of materials that are not presently required for sample processing.

Some or all of the work cells (e.g., including both activity work cells and buffer work cells) may further include various types of multi-temperature storage, which are designed to provide optimal storage conditions for a wide variety of biological samples and reagents.

These storage units may be capable of maintaining and regulating multiple temperature zones, ranging from ultra-low temperatures of −80° C. to standard refrigeration temperatures of 4° C., and ambient room temperature. This capability allows different types of biological materials, each with its unique stability and storage requirements, to be preserved under ideal conditions. For instance, nucleic acids like DNA and RNA, which are often sensitive to degradation, may be stored at −80° C. to maintain their integrity over extended periods or may be stored at −20° C. over shorter periods. Meanwhile, enzymes and other reagents that require cold storage, but not ultra-low temperatures, may be kept at −20° C. or 4° C., so that they remain active and effective. The multi-temperature storage units may be integrated, with multiple temperature zones in a single standalone unit, or may be modular, with different temperature zones handled by separate standalone units, to allow for expansion based on the storage needs of a given work cell. In an aspect, the modular design of the multi-temperature storage units allows them to be easily integrated into existing laboratory setups and scaled according to the needs of the laboratory. Additionally, they may be configured to accommodate a wide range of sample types, from relatively “whole” samples (e.g., blood, urine, saliva, fecal samples) to components thereof, to purified nucleic acids and reaction mixtures.

In an aspect, access to the multi-temperature storage units may be facilitated by a robotic component integrated into the work cell. These robotic components may be equipped with control mechanisms and sensors, enabling them to navigate within the storage units. Additionally or alternatively, the multi-temperature storage units may be communicatively coupled with the system 100 such that requests to make available particular samples or materials are received from the appropriate layer of the hierarchical software architecture 100 and the robotic components may be equipped with mechanisms and programming to retrieve the requested samples and materials as presented. When a sample or reagent is needed, the robotic component may receive instructions from the second software layer, which may identify the location of the required item within the storage unit. The robotic arm may then move to the designated storage compartment, e.g., using its sensors to accurately position itself and retrieve the sample or reagent without compromising its integrity. In an aspect, the robotic arms may be designed to handle the diverse environmental conditions within the multi-temperature storage units. For instance, when accessing samples stored at −80° C., the robotic arm may be capable of operating in ultra-low temperatures, ensuring rapid and reliable retrieval. Similarly, the robotic arm may be capable of operating with items stored at −4° C. or room temperature. While large temperature differences between storage units are described in this example, it is considered that at least in some work cells, the temperatures of storage units within a work cell may not vary as widely, or at all. In an aspect, the robotic arm may adjust its handling techniques to accommodate different storage conditions. In an aspect, some or all of the multi-temperature storage units may be equipped with automated doors or hatches that the robotic arm can open and close (or that may be configured to automatically open or close in response to sensing that the robotic arm is approaching or in response to receiving requests for samples or materials stored therein). These doors may be synchronized with the robotic arm's movements to minimize the exposure of stored items to external environmental conditions, thereby preserving their stability.

In an aspect, inventory tracking and management within the laboratory system may be used for maintaining the efficiency and reliability of the system as a whole. This may be achieved through a combination of various technologies and systematic processes designed to promote control over the storage and retrieval of biological samples, reagents, and consumables. In an aspect, each sample or reagent stored within the multi-temperature storage units, buffer work cell, and laboratory system generally, may be assigned a unique identifier, e.g., in the form of a barcode, QR code, or RFID tag. These identifiers may be scanned and recorded (e.g., to LIMS) at the time of storage, thereby logging details such as one or more of the sample type, patient identifier associated with the sample, sample batch identifier, age of the sample, storage temperature, quantity, and location within the storage unit. In an aspect, the status of items within the storage units may be monitored, and updates may be recorded in substantially real-time. For instance, when a sample or reagent is placed into a storage compartment, the robotic arm may scan its identifier and record the storage conditions and location to LIMS. As further described herein, LIMS may be queried by the top software layer to determine the availability and status of specific samples or reagents at any given time. In an aspect, as samples and reagents are retrieved for use, the samples and reagents may be scanned again, updating the inventory status in LIMS to reflect the removal of the item from storage. If a sample is returned to storage after partial use, its identifier may be scanned once more, and the updated information, such as remaining quantity and new storage location, may be recorded in LIMS. This dynamic tracking capability ensures that the inventory records are accurate and up-to-date.

In an aspect, the work cells may be situated along a conveyance platform, which connects work cells and facilitates the movement of samples throughout the processing pipeline. The conveyance platform may be controlled by the top software layer, which ensures that samples are transported efficiently and accurately between different stages of the workflow. In an aspect, the conveyance platform may take a variety of different forms. For instance, the conveyance platform described throughout this disclosure is a centralized motion platform that includes a conveyor belt or magnetic track that is configured to support and transport samples between different work cells. For example, the conveyance platform may be equipped with pucks, or other carriers, which are moved along the belt or track and are designed to transport a variety of sample containers (e.g., well plates, petri dishes, tubes, other laboratory vessels or specialized carriers depending on the nature of the sample and the requirements of the specific workflow, etc.) or other materials. These pucks may be configured to maintain the stability of the samples during transit, inhibiting spills, contamination, or mishandling. In an aspect, the design of the pucks may be customized to accommodate different types and sizes of samples, ensuring compatibility with the diverse needs of laboratory processes.

Beyond conveyor belts, other potential types of conveyance platforms may be utilized in lieu of, or in addition to, conveyor belts that are employed based on the specific requirements of the workflow and laboratory setup. For instance, one alternative is a robotic system, which may pick up and relocate samples with high precision. Autonomous robots or robotic arms may navigate paths and handle samples with care. In another aspect, a track-based shuttle system may be utilized, where autonomous shuttles move along fixed tracks to transport samples. These shuttles may be programmed to follow specific routes, stopping at designated work cells to deliver and pick up samples. The flexibility of this system may allow for dynamic routing and efficient management of multiple samples simultaneously. In yet another aspect, a pneumatic tube system may be leveraged to transport small, sealed containers quickly over longer distances within a facility. This system may utilize compressed air to propel tubes through a network of pipes, delivering samples rapidly and reliability to various destinations. In another aspect, a drone system may be used to transport carriers, samples, or materials between, e.g., a central storage unit and the work cells. Suitable systems may be, e.g., free-standing, or may be mounted to one or more of the floor, ceiling, or wall. In an aspect, each of the foregoing types of conveyance systems, or others, may be selected and utilized based on factors that are appropriate for the type of workflow, e.g., the layout of the laboratory, the nature of the samples being processed, and/or the specific workflow requirements. In an aspect, each of the foregoing types of conveyance systems may be implemented alone or, alternatively, may be used in combination if appropriate.

In an aspect, the system may contain a central repository that serves as the primary storage and staging area for samples and reagents before they are conveyed by the conveyance platform to work cells and processed. Upon arrival in the central repository, samples may be accessioned, cataloged, and stored in the central repository, which may be equipped with one or more temperature-controlled storage units capable of preserving a plurality of samples at optimal conditions. In an aspect, when a sample is scheduled for processing, it may be retrieved (e.g., using one or more robotic components) and transported by the conveyance platform from the central repository to a designated work cell (e.g., the first work cell in the workflow). In other aspects, the central repository may serve as long term storage for reagents and reserve amounts of samples after primary processing is complete. Samples may be accessioned and processed immediately after receipt at the library.

Referring back to FIG. 1, the top or first software layer 10, also known as the “Manufacturing Execution System (MES)” or “orchestrator,” serves as the central command unit of the entire system. This layer is primarily focused on managing the overall flow of samples and materials between different work cells, ensuring a seamless and efficient process from start to finish. It acts as the high-level decision-maker, utilizing comprehensive context data such as assay protocols, timing requirements, system component status, and inventory levels to make informed decisions about sample handling and routing. In an aspect, the MES may be configured to ensure that all necessary materials and instructions are present for the lower-level execution layers to function without interruption. By doing so, it maintains a steady and controlled flow of samples through the system, mitigating bottlenecks and optimizing throughput. This layer may be configured to handle hundreds, thousands, or millions of individual samples throughout the process, being apprised of their progress and dynamically adjusting the workflow as needed to accommodate the varying workloads, system status changes, and unexpected disruptions. Moreover, the MES may be configured to operate with a high degree of autonomy, thereby reducing the need for human intervention in sample management. In an aspect, the MES may coordinate the deployment of samples to multiple work cells, enabling them to process different samples simultaneously. For instance, while one work cell may be engaged in DNA extraction, another may be performing PCR amplification, and yet another may be conducting sample analysis.

It is important to note that the MES does not exert control over the specific processing steps occurring within each work cell and the tasks and operations performed by each instrument, but rather, coordinates the flow of samples and materials among work cells to ensure that each work cell timely receives the samples and resources it needs to perform its designated procedures. The MES knows what each work cell does (i.e., what the input and the output of each work cell will be), but not the individual steps performed within the work cell in order to produce the output. More particularly, the MES may be designed with a purposeful ignorance, delegation, and/or at least partial independence of the specific functionalities of the lower software layers. This architectural choice is integral to the system's efficiency, scalability, and reliability. More particularly, by not delving into the detailed operations of the subordinate layers, the MES can focus on high-level decision-making and workflow management without being bogged down by the complexities of individual step, task, and/or operation execution. Accordingly, although the MES layer may be configured to manage the overall flow of samples and materials through the system, it does not need to know the specific methodologies employed by the lower layers to execute these procedures.

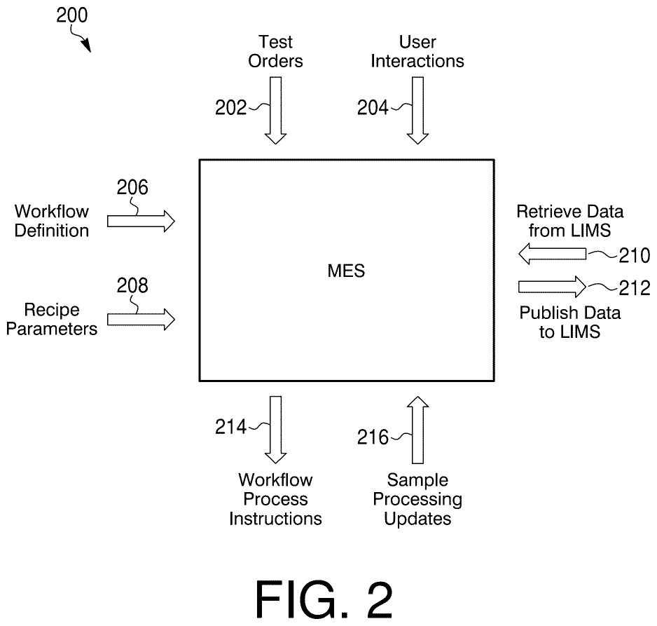

To effectively manage the flow of samples and materials throughout the system, the MES may reconcile a variety of different types of data. For instance, referring now to FIG. 2, diagram 200 illustrates the various types of inputs and outputs that are received and transmitted by the MES.

The MES may receive test orders 202, which are formal requests or directives to perform specific sample preparation, tests, or assays on a set of biological samples. These test orders 202 may originate from external sources such as clinicians, researchers, or other laboratory users who require specific analyses to be conducted on samples they submit. The components of a test order 202 may include one or more of: sample information (e.g., sample ID (a unique identifier for each sample)), sample type (blood, urine, tissue, etc.), quantity/volume (amount of sample provided, etc.), requested tests (e.g., test type (specific sample preparation, test, or assay to be performed)), priority level (urgency of test such as routine or urgent), special instructions (any special handling or preparation instructions required, etc.), client information (e.g., client ID (identifier for the person or organization requesting the test)), contact information (details for communication and reporting results, etc.), and/or submission date and deadline (e.g., date of submission (when the test order was placed), required completion date (when the results are needed), etc.). Test orders 202 may be used by the MES to determine and, in some instances, to prioritize the workflows that need to be executed. More particularly, the MES may take the information from these orders and translate them into actionable workflow process instructions.

The MES may be subject to various interactions 204 from users that may be provided to manage, monitor, and adjust laboratory workflows. These interactions may be important for ensuring that the system operates efficiently and effectively, even in an autonomous environment. Users, who may include laboratory technicians, researchers, quality control personnel, etc., may interact with the MES through a user interface that allows them to input data, receive updates, and make adjustments to ongoing processes. In an aspect, users may input various types of data into MES, such as new test or processing orders, sample details, reagent batch information, etc. Additionally or alternatively, users may manually adjust workflow parameters based on the characteristics of a sample batch and/or in response to receipt of real-time processing data. For example, users may change the temperature settings for an incubation step or modify the duration of a centrifugation process to optimize results. Users may also manually modify the work cells available for processing samples such as to temporarily disable work cells so that maintenance, repairs, or restocking can be performed. Additionally or alternatively, in cases where automated decisions need human intervention, users may override the system's actions, which may be necessary during unexpected events or when special handling of samples is required. Other types of user interactions with the MES, not explicitly discussed here, may also be provided.

The MES may utilize the workflow definition 206 and recipe parameters 208 to construct the workflow process, which may define the set of instructions and procedures designed to guide the processing of a sample or a batch of samples through various stages of analysis or production. With respect to the former, the workflow definition 206 refers to the high-level description and organization of the sequence of steps required to complete a specific laboratory process or achieve an overall goal. For instance, the workflow definition 206 may delineate that in a DNA extraction portion of the workflow process, the steps may include sample lysis, DNA binding, washing, and elution. In an aspect, the workflow definition 206 may also specify the dependencies between steps, indicating which steps must be completed before others can begin. This ensures that the workflow proceeds logically and that prerequisite conditions are met.

With respect to the latter, recipe parameters 208 refer to the specific instructions, conditions, and settings that define how a workflow should be executed. More particularly, these parameters may provide the detailed operational guidelines necessary for tailoring the execution of each step in the workflow to meet the particular requirement of different products, samples, or experimental conditions. Accordingly, while the workflow definition 206 outlines the high-level sequence of steps, the recipe parameters 208 ensure that each step task within these steps is performed with precision and according to specific criteria. For example, in a DNA extraction workflow, the recipe parameters may influence: temperature settings, reagent volumes, centrifuge speed, elution volume, and quality control parameters. In an aspect, the workflow definition 206 and the recipe parameters 208 may be pre-configured and stored within the MES if the system is designed to perform the same standardized process on each sample. Additionally or alternatively, if the system is capable of handling various sample types or executing different processes, the workflow definition 206 and the recipe parameters 208 may be received when new samples are introduced, allowing the MES to tailor the workflow process according to specific test orders and sample characteristics.

In an aspect, the MES may retrieve data 210 published to LIMS, e.g., by the work cells, to inform and guide the execution of workflows. The MES may rely on this data to make informed decisions, optimize processes, and ensure that the workflow process is effectively executed. For instance, the MES may access various types of data, including: sample history data (e.g., historical data on the sample, including previous processing steps and any prior results), reagent availability (e.g., information on the availability and status of reagents, potentially including one or more of batch numbers, expiration data, storage conditions, usage rate, current reagent volumes, reagent volumes used, etc.), equipment status (e.g., data on the availability, calibration status, and, processing efficiency of the work cell, maintenance history or instruments and equipment), and current process status (e.g., real-time data on the current status of workflow processes, including which steps have been completed and any deviations or issues that were encountered). In an aspect, the MES may be configured to access LIMS continuously, periodically (e.g., every minute, hour, etc.), or in response to predetermined events (e.g., prior to each downstream instruction transmission, in response to receiving an alert, etc.).

In the converse, the MES, along with each of the other software layers, may be configured to publish data 212 corresponding to sample processing and workflow progress to LIMS. By ensuring that relevant data is accurately recorded and accessible, this interaction supports traceability, compliance, quality control, and future analysis. The MES may publish various types of data to LIMS, including one or more of: workflow process information for a sample or sample batch, sample data (e.g., sample ID/barcode information, sample batch characteristics including sample type and number of samples in the batch, etc.), step completion updates (e.g., information on the completion of each workflow step, including timestamps and details of the specific tasks performed), end-point data (e.g., final results of the completed workflow), and the like.

By leveraging the workflow definition 206 and recipe parameters 208 to form the workflow process constructed to address the test order 202, and by further considering modifications to the workflow process provided by user interactions 204 with the MES and various information associated with the system retrieved from LIMS 210, the MES may generate and transmit workflow process instructions 214 to one or more components and/or system layers to initiate sample processing. In this regard, the MES may identify an appropriate activity work cell to begin the sample processing and may transmit instructions to the conveyance platform to initiate the transport of the sample to the designated work cell. These instructions may include one or more of: the current location of the sample, the destination of the work cell, and the optimal path for transport (e.g., taking into account real-time data about the system's operational status, such as the position of other samples or materials resident on the conveyance platform, work cell status, and other potential obstacles). Responsive to receiving these instructions, the conveyance platform may facilitate the transport process to the designated work cell. Additional details regarding work cell selection and conveyance platform configuration are further elaborated upon herein.

In one aspect, the MES may directly communicate with the scheduler associated with the designated work cell. For instance, the MES may transmit, to the relevant scheduler, a variety of information including one or more of: i) an indication that a new set of samples is being sent to that work cell for processing, ii) identification information associated with those samples (e.g., sample barcode IDs), and, in some aspects, iii) a subset of the workflow process instructions (e.g., including the relevant steps and tasks that need to be performed by the work cell for the type of sample) that are associated with the stage of sample processing that the work cell is configured for. In other aspects, the scheduler may always process the samples according to the same workflow process instructions, and so the workflow process instructions may not be transmitted from the MES to the scheduler. In an aspect, the scheduler, upon detection that a sample set has arrived at the work cell, may verify that the newly arrived samples correspond to the new processing request received from the MES (e.g., by scanning a barcode (or other identifying indicia) associated with the newly arrived samples and comparing the barcode information to the sample identification received from the MES transmission). The scheduler may then facilitate sample processing in accordance with the workflow process instructions received by the MES. In other aspects, verification may just involve the scanning of a barcode or other identifying indicia without comparing the scanned information to any other data received from the MES, or elsewhere.

In another aspect, the communication between the MES and the relevant scheduler may be simpler. For instance, the MES may simply transmit an indication to the scheduler that new samples will be arriving for processing, along with the relevant sample information. The scheduler, being configured to conduct specific steps of a stage of the workflow, may automatically initiate sample processing responsive to verifying that newly arrived samples match the sample information for the new processing request provided by the MES. Alternatively, in another aspect, the scheduler may be configured to automatically initiate sample processing responsive to simply scanning the barcode or other identifying indicia associated with the received samples.

In yet another aspect, the MES may have no direct communication with the scheduler. For instance, the MES may encode sample ID information into a barcode or other identifying indicia and facilitate transport of the samples to the relevant work cell using the conveyance platform. Alternatively, in another embodiment, no encoding of sample ID information may occur by the MES. Rather, samples may be placed in trays that include pre-assigned ID information, e.g., in the form of barcodes. As the tray moves from the central storage to a work cell (e.g., via the conveyance platform), a scanner may be configured to read the ID information, automatically logging the sample's entry into the workflow. Upon arrival at the work cell, the scheduler may detect that new samples have arrived, scan them, and utilize preconfigured knowledge of the steps that the work cell was configured to perform to execute the sample processing.

The MES may receive sample processing updates 216 from one or more of the downstream layers (or from LIMS after the downstream layers publish the sample processing updates 216 to LIMS). The MES may utilize these updates to facilitate decisions that optimize the processing of samples throughout the system. For instance, in an aspect, the MES may receive an indication from a relevant scheduler when a new sample batch is received at a work cell. In some aspects, this transmission may include a confirmation that all samples in a sample batch were positively identified. In other aspects, a transmission may not be sent from the relevant scheduler when a new sample batch is received. In another aspect, the MES may receive a transmission from the relevant scheduler that the processing of all steps and tasks associated with a sample are complete, or, in other words, that a sample has been output from the respective activity work cell. In another aspect, the MES may receive transmissions from a scheduler that provide an indication of instrument status (e.g., including operational state such as online, offline, in error, etc.), maintenance alerts (e.g., notifications of instruments requiring maintenance, calibration, or repairs, which may be triaged as immediate concerns or future considerations), reagent and consumable levels (e.g., the current stock levels of critical reagents and consumables (or indications of how much has been used), low stock alerts, expiration alerts, etc.), and the like. In an aspect, the sample processing updates may be received without explicit communication with any scheduler layer. For instance, the MES may be apprised of instrument health or status, inventory considerations, and sample processing updates by accessing information in LIMS that the scheduler or instruments within the work cell have published to LIMS. Based on the updates received, the MES may make various dynamic adjustments to the workflow. For instance, if an MES is apprised that an instrument is offline for maintenance, the MES may reroute samples to an alternative work cell with similar capabilities to ensure that processing continues despite required maintenance or other disruptions. As another example, the MES may initiate automatic replenishment protocols responsive to receiving indications that reagent and/or consumable levels are low.

The second software layer, also known as the “scheduler,” functions as the step executor within the hierarchical software architecture, shepherding the samples through the various steps associated with a sample stage within an individual work cell. In general, the scheduler's primary role is to map the necessary steps to the available resources within each work cell, ensuring that the workflow process associated with the work cell is completed efficiently. In an aspect, each scheduler, or scheduler thread, may be mapped to a robotic arm that is resident within each work cell, to the work cell itself, or to a work cell type. This relationship may facilitate precise and efficient execution of tasks by leveraging the scheduler's capacity to manage workflows and the robotic arm's ability to perform physical operations. More particularly, each scheduler may be responsible for managing and arranging for the execution of steps within a specific work cell, which may involve identifying the instruments that are configured to execute the tasks in furtherance of each step. The robotic arm may be utilized to execute specific tasks and/or to ferry the samples between the instruments in furtherance of those steps. For instance, the scheduler may provide the robotic arm with instructions, such as the specific movements required to transport samples within the work cell, the timing of these movements, and the exact locations for placing or retrieving samples. This coordination may enable the robotic arm to perform complex operations with high precision. For example, to complete a series of steps in a DNA extraction stage, the scheduler may instruct the robotic arm to pick up a sample plate from an incubator, transfer it to a centrifuge, and, once centrifugation is complete, then move it to a liquid handler for reagent addition. In an aspect, the scheduler may be configured to substantially continuously or periodically monitor the progress of steps and the status of the robotic arm and other instruments within each work cell. If any issues arise, such as a mechanical fault or an unexpected delay in a task, the scheduler may dynamically adapt by identifying other available instruments that may be configured to complete the necessary tasks in furtherance of each step.

Additionally or alternatively to the foregoing, a single scheduler may be responsible for managing several work cells that perform the same overall function or similar tasks. This configuration may be beneficial for scaling operations, as it allows the system to handle larger volumes of samples while maintaining consistency across multiple work cells. In an aspect, the scheduler may allocate steps and tasks based on the current load, availability, and efficiency of the work cells it oversees. By overseeing multiple work cells, the scheduler may coordinate resource sharing, such as redistributing reagents or samples to underutilized work cells.

In an aspect, the scheduler may be configured to manage multiple batches simultaneously within a single work cell. More particularly, the scheduler may be configured to process multiple sample batches at various stages of the workflow, ensuring that each batch receives the appropriate level of attention and resources. This concurrent processing not only enhances efficiency by reducing the downtime for any given sample and/or instrument, but also allows the system to handle high volumes of samples without compromising on the precision and accuracy of individual tasks. Similar to the MES layer, the scheduler may be configured to operate independently of the overarching context of the workflow, focusing solely on the execution of specific steps within the work cell. This deliberate isolation allows it to optimize its operations without being burdened by the complexities of the entire system.

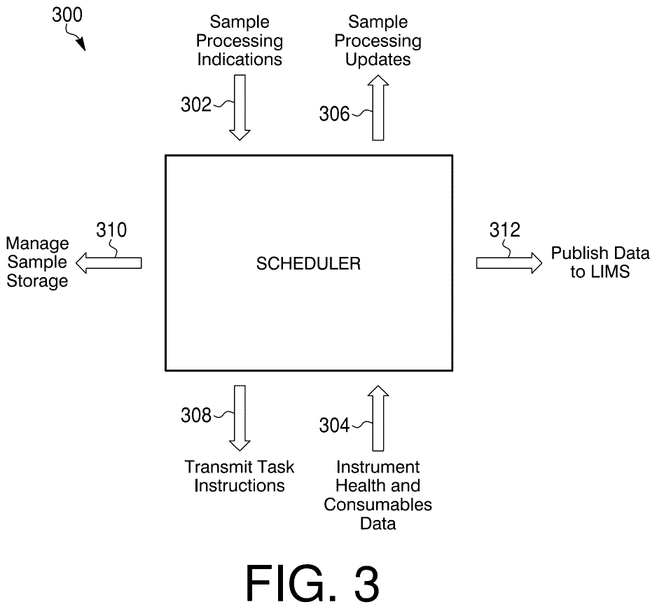

To effectively manage the sample through the execution of steps in a particular stage of the workflow, the scheduler may reconcile a variety of different types of data. For instance, referring now to FIG. 3, diagram 300 illustrates the various types of inputs and outputs that are received and transmitted by the scheduler.

In an aspect, the scheduler may receive sample processing indications 302, which act as the initial trigger for the scheduler to commence the processing of new samples. As previously discussed above in association with FIG. 2, in one aspect, these indications may be received from the MES in the form of detailed instructions associated with a particular portion of the workflow process. For instance, the instructions may include key information such as sample identification details and specific workflow process instructions tailored to the type of analysis or assay required. In another aspect, the sample processing indications 302 may be inherently deduced. For instance, a scheduler that identifies that samples have arrived at a work cell may contain preconfigured logic to automatically begin processing of those samples according to a stored workflow (e.g., which may delineate steps associated with a stage of an assay). Stated differently, the act of detecting that a sample has arrived at the work cell corresponds to the sample processing indication.

In an aspect, the collection and monitoring of instrument health and consumables data 304 promote maintenance of an efficient and reliable work cell. This data, gathered from various instruments within a work cell, may provide the scheduler with real-time insights into the operational status and readiness of the equipment. Instrument health or status data may include information on the current functioning state of each instrument, such as whether it is online, offline, or in an error state or reduced efficiency state, as well as details on any maintenance requirements, calibration needs, or recent performance issues. This information may allow the scheduler to make informed decisions about task allocation and to anticipate and address potential disruptions before they affect the workflow. Additionally, consumables data tracks the availability and levels of critical reagents, buffers, and other materials necessary for sample processing. Alerts for low stock levels, expired reagents, or other consumable-related issues, and/or requests to replenish the same, may ensure that the work cell may operate without interruption.

In an aspect, the scheduler may communicate with the MES to provide sample processing updates 306. These updates may provide real-time information about the status and progress of sample processing within the work cell. In an aspect, the scheduler may be configured to transmit these updates at various stages of the workflow, and these updates may include various types of information, including one or more of confirmations of sample receipt, detailed progress reports on the completion of each processing step, and the operational status of instruments involved in the workflow. Additionally, in some aspects, the updates may highlight any maintenance alerts, such as instruments requiring calibration or reagents running low. Through these updates, the MES may be able to make informed decisions about subsequent workflow steps, resource allocation, and any necessary adjustments to ensure smooth and efficient progression of sample processing. In other aspects, the scheduler may only communicate to the MES once a batch of samples is output from the work cell as completed. Other information, such as that described above, may be published to the LIMS, and the data may be retrieved from LIMS by the MES.

In an aspect, once the scheduler receives the sample processing indications and determines the necessary steps for each sample, it may execute these steps by allocating task-specific instructions 308 to the relevant instruments involved in each step. For instance, these instructions may include specific parameters and protocols for each task, such as one or more of volumes for pipetting, duration, and temperatures for incubation, speeds and durations for centrifugation, and any other operational settings required for the step. In some aspects, the instructions may be tailored to the capabilities and current status of each instrument, ensuring that the tasks are executed within the present performance range of the instruments. It is important to note that the scheduler itself may not oversee control of the execution of any particular task. Rather, after providing the sample to the relevant instrument, the scheduler waits until the instrument has completed the operations needed to fulfill the task, before resuming control to transfer, if necessary, the sample, or cause the sample to be transferred (e.g., via the robotic arm) to the next instrument. In an aspect, within a work cell, the scheduler may be responsible for orchestrating the movement of samples to various instruments in a specific order, so that each instrument performs its designated task in furtherance of a specific step. By coordinating the sequential handoff of samples between instruments, the scheduler facilitates the smooth progression of tasks, allowing the work cell to efficiently complete a complex multi-step process.

In an aspect, the scheduler may manage the storage of samples 310 within a work cell. This process ensures that samples are maintained in the appropriate conditions when they are not actively being processed, thus preserving their integrity and quality for subsequent steps. In this regard, the scheduler may be responsible for coordinating the placement of samples into appropriate storage units, such as multi-temperature storage compartments, which may include ultra-low temperature freezers, regular freezers, refrigerators, or ambient storage units, depending on the specific requirements of the samples. In an aspect, the scheduler may manage sample storage by issuing instructions to the robotic arm to transport the samples to and from the storage units. This may involve determining the correct storage conditions based on the sample type and the stage of the workflow. For instance, certain biological samples, like DNA or RNA, may require storage at −20° C. to prevent degradation, while others may only need refrigeration at 4° C. In an aspect, the scheduler may be configured to continuously or periodically monitor the status of the storage units, ensuring that they maintain the correct temperatures, within an acceptable margin of error, and that there is sufficient capacity for incoming samples. The scheduler may also be configured to track the location of each sample within the storage units to enable the efficient retrieval of samples when they are needed for the next stage of processing, thereby avoiding delays and facilitating a smooth workflow.

In an aspect, the scheduler may record and publish data 312 to LIMS related to, e.g., sample processing, instrument performance, and workflow progression. The scheduler continuously or periodically collects and updates information as it manages the execution of tasks and steps within each work cell. This data may include e.g., one or more of: details of each operation performed (e.g., such as the time and date of task initiation and completion), the specific instruments used, the conditions and parameters under which each step was executed, any deviations or errors that were encountered, instrument health data, consumable usage or levels, reagent usage or levels, sample storage data (e.g., which samples are in storage, the designated storage compartment for each sample, the sample's location in the storage module, how long they have been in a storage module, etc.), and the like. By publishing this data to LIMS, the scheduler may ensure that all actions are accurately documented and are traceable. This comprehensive tracking may also support compliance with regulatory standards and facilitate better quality control and auditing processes. Additionally, keeping LIMS updated may enable the MES to access real-time updates on sample status, make informed decisions about workflow adjustments, and ensure that conditions are met for subsequent processing steps. Additionally or alternatively, in an aspect, the data stored in LIMS may be used for retrospective analysis, helping to identify trends, optimize protocols, and improve overall laboratory efficiency.

The third software layer 30, also known as the “instrument control layer,” exists at the instrument level and is responsible for the direct control and management of the specific tasks and operations performed by each instrument within the work cell. Unlike the higher-level software layers that handle workflow orchestration, the instrument control layer focuses on executing the specific tasks designated by the scheduler, or the tasks that it is dedicated to perform. These tasks may be broken down into detailed operations that the instrument must perform. For example, in a DNA extraction process, tasks may include pipetting specific volumes of reagents, mixing samples, incubating them at certain temperatures, or centrifuging at defined speeds. To execute each of these tasks, an instrument must perform one or more atomic operations that are executable by the instrument's hardware components.

In an aspect, the instrument control layer operates with a focused scope and does not need to understand the overall process or the sequence of tasks; instead, it concentrates solely on the specific operations that the instrument the instrument control layer oversees is tasked with performing. In an aspect, this narrow focus allows the instrument control layer to optimize the performance of individual instruments, ensuring that each operation is executed with accuracy. For instance, a robotic arm controlled by the instrument control layer may follow coordinates and timing to transport samples, avoiding the risk of human errors. Similarly, as another example, a pipetting instrument may dispense predetermined volumes of reagents from predefined supplies, adhering to predefined parameters to ensure reproducibility and consistency across all samples. The instrument control layer may also be configured to provide real-time updates to the LIMS. More particularly, as each operation is initiated, progresses, and completes, the instrument control layer records detailed data on the status and outcomes of these operations to LIMS. This recorded information may allow for tracking the progress of individual samples or batches of samples, promoting traceability, and facilitating quality control.

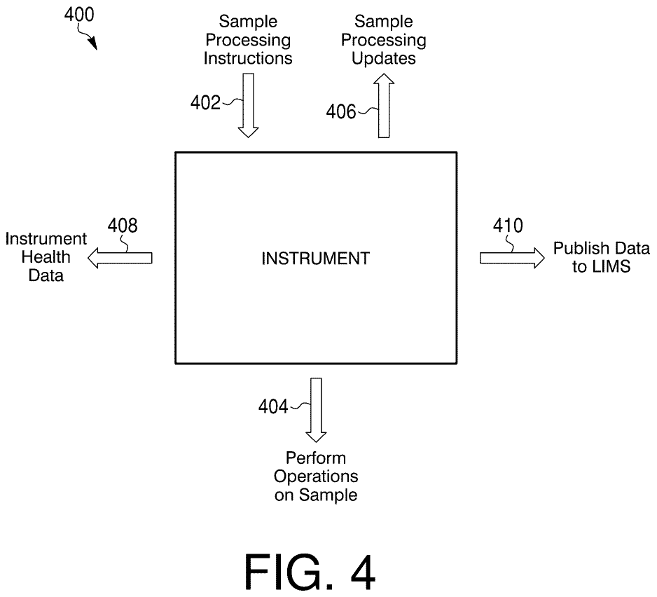

To effectively complete tasks in furtherance of the steps involved in a processing stage, the third software layer may reconcile a variety of different types of data. For instance, referring now to FIG. 4, diagram 400 illustrates the various types of inputs and outputs that may be received and transmitted by the instrument control layer.

In an aspect, the instrument control layer may receive sample processing instructions 402 from the scheduler. The instructions may be derived from the scheduler in the form of task indications that the instrument must complete. In an aspect, an instrument may be responsible for completing an entire task or series of tasks. In another aspect, the instrument may only be responsible for completing a portion of a task, the remainder of which may be completed by another instrument. In other aspects, the sample processing instructions 402 may simply be that samples are ready for the instrument to process, and the individual instrument may be designed to process the same task or series of tasks for each sample batch it receives.

In an aspect, the instrument control layer may translate the high-level workflow requirements into actionable commands for the hardware components of the instrument, which may manifest as operations performed on the sample 404. Each instrument in the work cell may be designed to carry out operations, such as, e.g., pipetting, mixing, heating, cooling, and/or centrifuging, depending on the needs of the particular step and stage of the workflow. These operations may be typically executed through a series of automated actions controlled by the instrument control layer, which interprets the instructions and converts them into specific mechanical actions. These operations may be controlled and monitored to promote accuracy, precision, and consistency. In an aspect, sensors within the instrument may provide real-time feedback to adjust parameters dynamically, promoting optimal performance and avoiding errors.

In an aspect, the instrument may transmit sample processing updates 406 to the scheduler during the execution of tasks on a sample. These updates may serve multiple purposes, including informing the scheduler of the progress and status of sample processing and facilitating real-time tracking and transparency. In an aspect, as the instrument performs its designated operations, it continually or periodically generates data, e.g., about each operation and task completion, any deviations from expected performance, and the current state of the sample. This data may be encapsulated in processing updates, which may include details such as timestamps of task initiation and completion, quantitative measurements (e.g., volumes of reagents used, temperatures maintained), any anomalies or errors encountered. In an aspect, these updates may be transmitted at the conclusion of each task, at the conclusion of a series of tasks associated with a step, at the conclusion of all tasks for a particular sample batch, or at predetermined intervals. By transmitting these updates to the scheduler, the instrument may ensure that the system remains synchronized and that any necessary adjustments may be made promptly to optimize workflow efficiency. Additionally, the scheduler may rely on these updates to make informed decisions about the execution of subsequent steps in the process. In other aspects, these details may be published to LIMS instead of or in addition to the scheduler, and if only published to LIMS, the output to the scheduler may simply be when the tasks for a sample batch are completed, or if there is an error or maintenance issue.

In addition to sample processing updates 406, the instrument control layer may also transmit instrument health data 408 to the scheduler or LIMS. The instrument health data may include, e.g., one or more of detailed diagnostics, such as error codes, sensor readings, and calibration status. If the instrument detects any anomalies or malfunctions, it may send alerts specifying the nature and severity of the issue. For instance, if a robotic arm is experiencing increased resistance in its movement or a pipette is delivering inconsistent volumes, these deviations may be logged and reported. The proactive communication may enable the scheduler to reallocate tasks to other functioning instruments or work cells, reducing downtime and maintaining the workflow's continuity.

While performing the sample processing tasks and operations, the instrument control layer may publish data to LIMS 410. This data may include records of tasks performed by the instrument, such as timestamps for the start and completion of tasks, the specific operations executed, and the results obtained from these operations. By systematically documenting each step of the sample processing workflow, LIMS serves as a centralized repository for data, ensuring that actions performed on a sample are tracked and recorded. In addition to operational data, LIMS may also receive updates on one or more of instrument health, maintenance requirements, and consumable levels. The MES may access this information in LIMS, which it may utilize to help organize the workflow process.

The fully automated system described herein is designed with a high degree of flexibility and adaptability. In some aspects, different work cells may be able to perform specific steps of a process differently based on the type of sample being processed. This capability ensures that each sample type receives the most appropriate and effective treatment, tailored to its unique properties and composition, while still achieving consistent and standardized outputs. For instance, biological samples such as blood, urine, saliva, and tissue each have distinct properties that may necessitate specialized processing methods. For example, blood samples may require lysis of red and white blood cells, followed by separation of plasma and extraction of nucleic acids from the cellular components. In contrast, urine samples, which are typically less dense compared to blood samples, have a different composition of solutes and cells, and may need different concentration and purification steps to isolate the desired analytes. To accommodate these differences, work cells may be equipped with specialized instruments, reagents, and protocols that are designed to handle the specific challenges posed by each sample type. In other aspects, the system may include multiple different work cells for achieving the same step (e.g., DNA extraction or cytosine conversion for methylation analysis via bisulfite conversion or enzymatic conversion), and some of the work cells may be appropriate for a certain sample type (e.g., blood samples), while other work cells are appropriate for a different sample type (e.g., urine) or workflows (e.g., one ending with a cancer detection report vs. one targeting minimal residual disease analysis). The MES may assign a sample of a given type to a work cell that is capable of processing that sample type. Accordingly, the MES may assign each sample to a work cell that is appropriately equipped to handle its specific processing needs, ensuring that the steps taken within each cell are optimized for the sample type.

At the same time, there may be steps that are unified across sample types for a given workflow. As an example, a workflow may be configured for cell free DNA (cfDNA) sequencing and analysis. cfDNA can be extracted from a variety of sample types including blood and urine. Blood and urine samples may begin their workflow differently, however the output of a DNA extraction work cell may be the same for both blood and urine originating samples. From the DNA extract pathway onwards, the MES may be configured to treat the blood and urine originating samples the same using the same work cells. As another example, cfDNA analysis may include methylation or targeted methylation analysis or whole genome sequencing and analysis. Therefore, after a DNA extraction work cell, samples may be selectively directed to partitioning-based partitioning work cells, bisulfite conversion work cells, work cells to introduce probes to target specific regions of the genome, or to pre-amplification work cells to prepare for whole genome sequencing. As can be seen, the flexibility and modularity afforded by the MES enables for different types of samples and different assay outputs to be processed together around a similar core of work cells in a fully automated system.

In an aspect, despite the variations in processing methods across different work cells, the system may be designed to ensure that the final outputs are consistent and meet the required quality standards. For example, the goal of DNA extraction may be to obtain purified DNA that is suitable for downstream applications, such as sequencing polymerase chain reaction (PCR) or sequencing and analysis of a variety of types (e.g., whole genome sequencing, methylation-informed sequencing, fragmentation analysis). Whether the DNA is extracted from blood or urine, as in the example above, the system ensures that the purification process yields DNA of sufficient quantity and quality. This may be achieved by tailoring the intermediate steps to the sample type while maintaining control over the final output criteria. As a result, the system may produce standardized outputs from varied inputs, making it versatile and efficient. Although the ability to handle different sample types is described herein, it is acknowledged that in other aspects, the system may only receive one sample type, and thus may not be configured to receive multiple different sample types.

In an aspect, system 100 may have the capacity to undergo improvements and adjustments to instruments and hardware components while remaining “online,” or operational. This capability may allow for continuous system functionality and minimal disruption to ongoing processes to maintain high throughput and efficiency in laboratory environments. In an aspect, when an instrument or hardware component requires maintenance, upgrades, or adjustments, the MES may dynamically reallocate tasks to other available work cells or instruments. For example, if a specific automated pipettor in a work cell needs calibration, the MES may temporarily route samples to an alternative work cell that is configured for a similar process flow, thereby ensuring that the overall workflow remains uninterrupted. Similarly, if a work cell is in need of maintenance or repair and needs to be taken offline for a period of time, then samples may be directed by the MES to alternative work cells of the same type so that sample processing may continue, albeit with potentially reduced capacity, while avoiding work stoppage of the entire system. This dynamic task reassignment may allow for real-time adjustments without halting the entire system.

Furthermore, the modular design of the system supports the addition or removal of hardware components without necessitating a complete shutdown. Specifically, new instruments may be integrated into existing work cells, and software updates may be deployed to enhance performance or introduce new functionalities. In an aspect, the scheduler layer, which manages steps within individual work cells, may incorporate these changes, updating its task mapping to include the new or modified components. For instance, given a scenario where a new type of reagent dispenser is introduced in a work cell to improve sample preparation, the MES or the scheduler may be apprised of the updated functionality associated with the work cell and may thereafter update the workflow processes to utilize the new dispenser. As another example, new work cells may be integrated into the system, e.g., to increase the system's capacity, or software updates may be pushed out to one or more work cells. The MES, which manages the allocation of samples to individual work cells, may incorporate these changes, updating its task mapping to include the new or modified work cell. For instance, given a scenario where a new work cell is introduced, the MES may be apprised of the new work cell or updated functionality associated with the work cell and may thereafter update the workflow processes to utilize the new or updated work cell. As such, multiple versions of the same kind of work cell (e.g., Extraction v1and Extraction v2) can operate together, with the MES being able to treat them as identical units so long as the input and output requirements are the same, or at least compatible.

### Buffer System

The system may contain a plurality of strategically placed buffer work cells, which may serve a role in enhancing the efficiency and continuity of the overall workflow. These buffer work cells may function as intermediaries within the overarching system, temporarily storing one or both of resources and samples to smooth transitions between different stages of the workflow. In traditional systems, the direct handoff of samples and materials from one processing step to another often leads to inefficiencies, such as delays or bottlenecks, particularly when a downstream process is not immediately ready to receive them, or when a new sample or replenished supplies are needed, but they have not yet been provided. The buffer work cells address this challenge by acting as holding areas that decouple the timing of upstream and downstream processes, allowing each stage to operate independently at its optimal pace. More particularly, since different assay steps may require varying amounts of time to complete, the buffer work cells mitigate the risk of one stage holding up the entire process.

In an aspect, by buffering resources and samples, these buffer work cells ensure that the system may continue processing without interruption, or with little interruption, even if certain stages of the workflow are momentarily delayed. For instance, if a downstream activity work cell is busy or requires maintenance, the buffer work cell may temporarily store incoming samples, avoiding a bottleneck that may otherwise halt upstream processes. Similarly, in an aspect, the buffer work cells may store critical reagents and consumables, so that these materials are readily available when needed, thus avoiding the downtime associated with waiting for supplies. Moreover, supplies can be delivered to the buffer work cell by a conveyance platform well in advance of the supplies being needed by the activity work cell downstream and without interrupting the distribution of samples. This capability is particularly important in high-throughput environments in which delay may significantly impact overall productivity.

In an aspect, the MES may determine when and how to allocate samples and resources to a buffer work cell. To facilitate this determination, the system may assess the current status of the workflow, including, e.g., one or more of the availability of downstream activity work cells, the readiness of resources, the availability of resources to allocate, the supply of resources at one or more work cells, the importance of a given work cell to the overall system, the availability of other work cells that are capable of performing the same function of a given work cell (and thus the redundancy in the system for that type of work cell), the time it takes for a work cell to complete its function, the current efficiency or capacity of a work cell, and the timing of ongoing processes. For instance, if an activity work cell is occupied, scheduled to undergo maintenance, or undergoing maintenance, the system may decide to temporarily store samples in a buffer work cell to avoid delays and maintain workflow continuity. Alternatively, if the activity work cell is available and ready to process a sample, the system may move the sample directly from the buffer work cell to the activity work cell without delay.

Another decision point may pertain to prioritizing the use of buffer work cells during peak processing times or when unexpected disruptions occur. The MES may evaluate whether to use a buffer work cell to hold samples or resources that are in high demand, balancing the need to avoid bottlenecks with the goal of maximizing throughput. More particularly, the MES may continuously or periodically monitor the status of all work cells, to determine how to handle fluctuating demands and avoid potential bottlenecks. For instance, during peak processing times, if multiple activity work cells are reaching their capacity, the MES may decide to temporarily store samples in buffer work cells to prevent the system from becoming overloaded. This proactive use of buffer work cells may help maintain a steady flow of samples and ensures that no single work cell is overwhelmed, thereby avoiding bottlenecks that could slow the entire workflow.

Additionally or alternatively, the MES may consider the storage conditions required for different types of samples or resources, ensuring that the buffer work cell can provide the appropriate environment to maintain sample or resource integrity. More particularly, to ensure that the samples remain viable and are not compromised during their time in storage, the MES may dynamically assess and match the conditions of the buffer work cells with the needs of the materials they are storing. Buffer work cells may be equipped with various storage environments, such as temperature-controlled compartments, humidity-regulated space, etc., for preserving specific types of samples. When determining where to store samples or resources temporarily, the MES may evaluate the specific storage conditions each type of sample or resource requires. If a sample, or materials needed by the activity work cell to process the sample, need to be stored at a particular temperature or under certain humidity levels to maintain its integrity, the MES may ensure that it is placed in a buffer work cell that meets those criteria.

In other aspects, the shelf life of a sample at a particular stage in processing or of a resource may be taken into consideration when determining whether and where to store that sample or resource in a buffer work cell based on how soon it may need to be acted upon by an activity work cell. If a sample is left at a buffer work cell too long, it may degrade, leading to compromised results or the need to initiate a reprocessing action. Accordingly, based on the remaining shelf life, the MES may determine the most appropriate buffer work cell to store a sample or resource. For example, if a sample has a short shelf life and needs to be processed soon, it may be placed in a buffer work cell that is located close to the next activity work cell in the workflow. This proximity ensures that the sample can be quickly retrieved and moved to the next processing stage without unnecessary delays. Conversely, if a sample or other material has a longer shelf life and is not immediately needed for the next step in a work flow, the MES may choose to store it in a buffer work cell that is slightly farther away or in a less prioritized location. This approach allows more urgently needed samples or resources, with shorter shelf lives, to occupy the buffer work cells that are closer to the activity work cells so that they are processed in a timely manner. In other aspects, if a sample has a short shelf life (i.e., be relatively less stable) after one step of the process is performed, the MES may determine that the relevant step should not be performed until a time when the subsequent step would be able to be performed by an activity work cell within the shortened shelf life time. For example, the relevant step may not be performed until the work cell performing the subsequent step has been replenished with the needed resources or would be available to process the sample once the relevant step is completed.

In still other aspects, a central storage unit of the system may be configured to maintain samples at a colder temperature compared to other local storage units or buffer work cells in the system. In some instances, it may be that a shelf life of the sample is shorter once the sample is moved from central storage to downstream locations in the system for processing.

Accordingly, in such aspects, the MES may only move a sample from central storage at a time when downstream steps in the process may be able to be performed within a shelf life of the sample once the sample is removed from sample storage. In some aspects, this may also or alternatively be true for certain reagents, which may be held in a main storage repository before being moved to local storage or buffer work cells within the system.

In an aspect, the system may also need to make dynamic decisions about when to retrieve samples or resources from buffer work cells for further processing. This decision may be influenced by a variety of factors. For instance, in one example, the decision may be influenced by real-time data on workflow progress. This may involve continuously or periodically monitoring the status of ongoing processes within the system. This real-time data may provide insights into the current stage of each sample or resource within the workflow, the availability and readiness of activity work cells, and any potential bottlenecks or delays.

As another example, in an aspect, the decision to retrieve samples may be influenced by the operational status of activity work cells. This status encompasses whether a particular work cell is currently active, idle, undergoing maintenance, or experiencing a malfunction. By monitoring the operational status of these work cells in real time, the system may make informed decisions about when to deliver samples and/or resources to or release samples and/or resources from buffer work cells. For instance, if an activity work cell is temporarily unavailable due to maintenance or an unexpected issue, the system may choose to hold samples in the buffer work cells until the activity work cell is back online and ready to resume processing, or the system may choose to move samples in the buffer work cells upstream of the temporarily unavailable activity work cell to a different buffer work cell that is upstream of an activity work cell that can perform the step of the work flow scheduled for the samples.

In yet another example, in an aspect, the decision to retrieve samples may be influenced by the shelf life of a sample or resource. The shelf life may refer to the period during which a sample or resources remains viable, stable, or effective for its intended use. In a laboratory setting, certain samples and reagents may have limited stability and may require timely processing to ensure accurate and reliable results. Real-time monitoring of the shelf life may enable the system to prioritize the retrieval of samples or resources that are approaching the end of their usable life, enabling them to be processed or utilized before they degrade or expire.

In yet another example, in an aspect, the decision to retrieve samples or resources from buffer work cells may be influenced by the availability of necessary reagents or tools required for subsequent processing steps. In a laboratory workflow, each step often depends on specific reagents or instruments being available and ready for use. If a buffer work cell stores samples awaiting further processing, the system may ensure that the necessary reagents or tools are readily accessible in the corresponding activity work cell before moving the samples.

In an aspect, the timing of retrieval may be determined so as to avoid idle time in activity work cells and to ensure that samples move through the system efficiently. More particularly, by coordinating when samples or resources are retrieved, the system may prevent activity work cells from experiencing idle time, which may help to maximize system throughput. In an aspect, the system may need to consider contingency plans for buffer work cells, such as rerouting samples if a buffer work cell reaches capacity or if an unexpected issue arises. For instance, if a buffer work cell reaches capacity due to an accumulation of samples, or if an unexpected issue arises (e.g., such as an equipment malfunction), the system may have contingency plans in place (e.g., to reroute samples or resources to other available buffer work cells or even directly alternative activity work cells that may be capable of performing the next required process step).

Further, in some aspects, the movement of samples through the system may be prioritized over the movement of resources through the system, e.g., in order to maintain high throughput and processing of samples. In this instance, resources may be moved through the system ahead of schedule, before they would typically be identified as needed by one or more work cells, if it is determined by the MES that samples will need to be moved through the system to that work cell or to a different work cell along that portion of the conveyance system at the time when the resources would typically be routed. Additionally or alternatively, an MES may move resources through the system later than normal, allowing the supply of resources to run lower than typical at a given work cell, in order to prioritize the movement of samples through the system to that work cell or to a different work cell along that portion of the conveyance system. In one example aspect, a work cell may even be allowed to run out of resources if the steps performed by that work cell are not immediately needed for the samples being processed at that moment and if samples need to be moved instead of resources at that time. This may also or alternatively occur if there was sufficient redundancy of that activity work cell type to handle the samples moving through the system if samples needed to be moved instead of resources at that time. The MES may prioritize the timely movement of samples through the system and may schedule the movement of resources around when samples are to be moved in order to promote high throughput of samples through the system.

### Resource Delivery to Local Storage

In the system, resources such as reagents, consumables, and other essential materials may be automatically delivered to local storage compartments that are strategically associated with each activity work cell, buffer work cell or a set of work cells (e.g., buffer work cell and one or more downstream activity work cell). These local storage compartments may be positioned in close proximity to the activity work cells, accessible by robotic features of the activity work cells, ensuring that the materials required for specific assay steps are nearby and quickly attainable. By having these resources proximately available, the system avoids the need for activity work cells to wait for materials to be fetched from a central inventory or from distant storage locations, which may cause delays in the workflow. Instead, as soon as a work cell requires a particular resource, it may be able to quickly and efficiently access the needed materials from its associated local storage. This setup may streamline the processing flow and reduce the risk of interruptions or bottlenecks caused by the unavailability of resources at critical moments.

In an aspect, the system may employ a monitoring mechanism that is configured to continuously or periodically keep track of the usage of reagents, consumables, and other essential materials stored in local storage compartments associated with each work cell. By integrating monitoring tools, e.g., such as scales placed under containers holding various materials, the system may be able to automatically measure the weight of the materials and detect when the weight falls below a predetermined threshold. When this threshold is reached, indicating that the amount of material is running low, the scales trigger a signal that prompts the system to send a request for additional supplies (e.g., as instructed by the MES).

In an aspect, while local storage compartments may provide immediate access to necessary resources, they may also be connected with a central inventory repository. This association promotes a continuous and automated supply chain feeding into local storage compartments, synchronized with the overall consumption patterns of the laboratory. In an aspect, when the local storage compartments reach or fall below a predetermined threshold level of resources, the MES may automatically trigger a replenishment protocol by which additional resources may be delivered from the central inventory to the local storage compartments. In an aspect, this replenishment process may be configured to occur during low-activity periods or scheduled maintenance windows, thereby reducing disruptions to samples traveling through the system according to ongoing assay workflows, e.g., as described above in reference to delivery to the buffer system.

In an aspect, shared local storage, if included in the system, may allow for more flexible allocation of resources. More particularly, buffer work cells may dynamically stock, and activity work cells may dynamically draw from, these storage units as needed, so that activity work cells aren't left waiting for important materials. This flexibility may be particularly helpful in high-throughput environments where the demand for reagents and consumables may fluctuate more rapidly. Additionally, by having a shared storage location, the coordination between activity and buffer work cells may be enhanced. The system may efficiently manage the flow of samples and materials, ensuring that each work cell receives the right resources at the right time range.

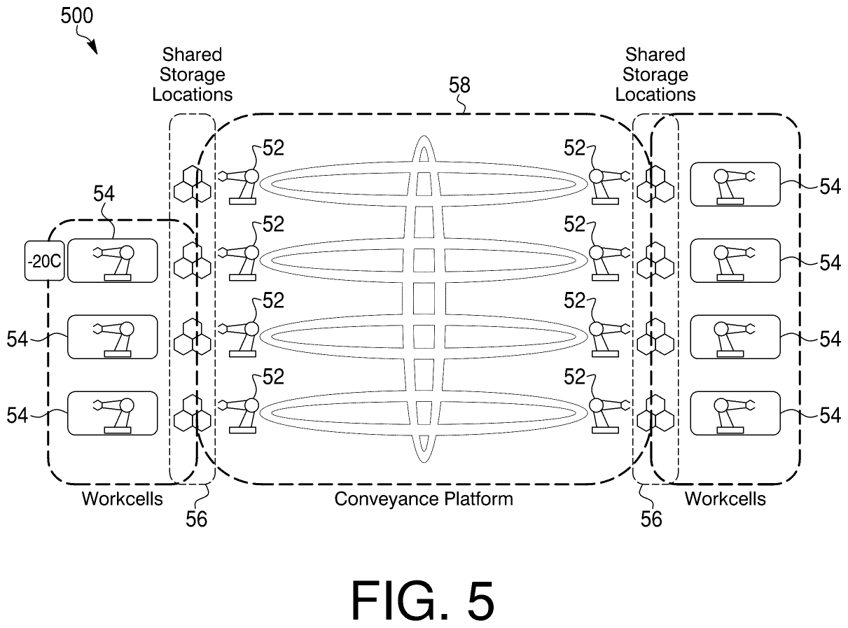

Referring now to FIG. 5, a diagram 500 is provided that illustrates the integration of various components, including work cells, shared storage locations, and a conveyance platform, all of which work together to optimize sample processing. In particular, diagram 500 illustrates how both activity work cells 54 and buffer work cells 52 may leverage a shared store location 56. More particularly, activity work cells 54 may require a supply of reagents, consumables, and other materials to perform their tasks, as these materials are consumed while performing the tasks assigned to them. By sharing local storage locations 56 with buffer work cells 52, they may be able to quickly access the resources they need without waiting for materials to be delivered from a central inventory. Similarly, additional benefits from shared local storage may include buffer work cells 52 accessing reagents and consumables needed for sample stabilization, temporary storage, or intermediate processing, if the buffer work cells 52 are so configured. By sharing these storage locations 56 with activity work cells 54, buffer work cells 52 can quickly move samples or materials in and out of storage, optimizing workflow continuity, and ensuring availability of the samples or materials to activity work cells. Additionally, shared local storage allows buffer work cells 52 to adjust their buffering capacity dynamically, depending on the current workflow demands and the availability of resources. In an aspect, the conveyance platform 58 serves as the central transport mechanism within the system. It facilitates the movement of samples and resources among central storage locations, work cells, and shared local storage locations. As discussed herein, the conveyance platform 58 may be dynamically controlled to prioritize the transport of samples and materials based on real-time workflow needs.

In the system, local storage compartments and buffer work cells may be flexibly allocated to activity work cells to optimize workflow efficiency and resource management. In some configurations, a local storage or buffer work cell may be allocated to an activity work cell on a one-to-one basis. This arrangement enables each activity work cell to have immediate and exclusive access to the consumables and temporary storage space it needs. In some aspects, this one-to-one allocation may be particularly advantageous for high-priority or high-throughput tasks, where dedicated resources may reduce the likelihood of delays caused by waiting for materials or storage availability. This arrangement may in some aspects be easier for the MES to track, as the use of resources by one work cell may be published to LIMS and may be monitored by the MES from LIMS in order to determine whether resources need to be replenished. Alternatively, the system may be configured so that more than one activity work cell shares local storage or buffer work cells. In this setup, a shared local storage or buffer work cell serves multiple activity work cells, dynamically providing resources as needed. This shared configuration may enhance flexibility and resource utilization, as it allows the system to adapt to varying demands across different work cells. For example, if one activity work cell is temporarily idle or processing a lower volume of samples, the shared storage or buffer work cell can redirect resources to another work cell that requires them more urgently. This shared approach promotes efficient use of space and materials and enables all activity work cells to be supported without the need for each to have a dedicated storage unit. In some aspects, a combination of shared and individualized buffer work cells and local storage may be used in the system.

In a non-limiting exemplary workflow, a biological sample may arrive at an activity work cell 54 for an initial processing step, such as DNA extraction. The necessary reagents may be accessed from the shared local storage 56, to facilitate starting of the process.

Once the DNA extraction is complete, the sample may be transferred to a buffer work cell 52 for stabilization and/or temporary holding. In one aspect, the buffer work cell 52 may draw reagents from the shared local storage 56 to prepare the sample for the next step in the workflow process, e.g., if the next step of the workflow is to be performed within that same work cell. In this setup, the necessary materials may be readily available and can be accessed to continue the workflow without substantial delay. The sample may then be moved to the appropriate portion of the activity work cell for amplification. Again, all necessary materials may be readily available in the shared local storage, allowing the process to continue without interruption or with little interruption. In contrast to the foregoing, if the next step of the workflow is to be performed by a different activity work cell, the system may move the sample first to that new work cell before drawing any required reagents or consumables from shared local storage 56 or buffer work cell 52 associated with the new work cell. This approach may be more efficient because each work cell now only draws the materials it needs at the time they are required. By doing so, the system may inhibit unnecessary reagent usage or wastage.

### Automatic Storage and Retrieval of Samples

In an aspect, the system may incorporate a sophisticated mechanism for the automatic storage of sample portions at predetermined stages of the assay process. This feature ensures that portions of biological samples or their byproducts are systematically preserved after predetermined steps in the workflow process (e.g., in the case of biological sample preparation, after significant steps such as sample preparation, DNA extraction, PCR amplification, sequencing preparation, etc.). During these steps, the system may identify excess materials or byproducts that may be stored for future use. For instance, after DNA extraction, a portion of the extracted DNA may be automatically aliquoted and stored. This aliquoting process may be controlled to ensure that sufficient material is preserved while maintaining the integrity and quality of the sample. More particularly, after the DNA extraction is complete, the system may automatically aliquot, or divide, the extracted DNA into two or more smaller portions. This aliquoting is controlled to guarantee that enough material is preserved in each aliquot to support potential future analyses or reprocessing, should the need arise. In an aspect, because the amount of a biological sample that is available may be variable and not fully predictable, a first aliquot may be allocated an amount determined to be needed to continue the workflow, while the second aliquot may be allocated any reserve amount of the sample available at the time. In an aspect, the storage process may be managed using integrated storage units that may be equipped with temperature-controlled compartments or multi-temperature compartments, capable of maintaining the required storage conditions for different types of samples. In an aspect, the excess materials may be stored in a buffer work cell and/or activity work cell's local storage. In an aspect, the excess materials may be stored in the central inventory repository. In a further aspect, the excess materials may be initially stored at local storage and later moved to the central inventory repository after it is determined that the excess materials are not likely to be immediately needed in local storage.

In an aspect, the predetermined stages may correspond to steps after which the resulting sample has a sufficiently long shelf-life and remains stable enough to allow for storage without risking substantial degradation. For example, after a purification step that isolates DNA, the resulting DNA is typically in a stable form that can be stored under appropriate conditions, such as refrigeration or freezing, without significant loss of quality or integrity over time. In another aspect, the predetermined stages may correspond to steps that achieve significant milestones in the processing of a sample, or result in a substantial change in its characteristics.

These milestones may involve critical transformations (e.g., extraction processes that separate a specific component from a mixture). At these points, the sample has likely undergone a key transformation that not only represents a checkpoint in the workflow, but also creates a product that has defined and stable characteristics. Storing the sample at these junctures allows the system to retain valuable intermediate products that may be revisited for further analysis or used to correct issues downstream without redoing the entire procedure.

In an aspect, the stored sample portions may be kept for a predetermined period of time, balancing the need for potential reprocessing of a sample with efficient resource management and storage use. This period may be dependent on one or more factors, e.g., the type of assay being performed on the sample, the stability of the biological sample, and/or a conventional timeframe for completing the entire assay process and reporting results. In an aspect, samples may be stored until the assay results have been confirmed and reported to the relevant stakeholders, such as researchers, clinicians, or patients associated with a given sample. This ensures that the samples are readily available for reprocessing if any issues arise during the downstream processing steps or if there is a need to validate or repeat certain assay results (e.g., as a result of a patient request, a sample within a batch fails a quality control protocol, issues with reagents were discovered during quality assurance, etc.). For example, if a DNA sequencing assay reveals an anomaly, the system may be configured to retrieve the stored DNA portion and reinitiate the sequencing process, promoting the achievement of accurate and reliable results without the need for new sample collection. Additionally or alternatively, in some aspects, the system may be able to determine whether to initiate the sequence process on another sample or not. For instance, if an error occurs during a task in a given work cell, the system may attempt to rectify the issue by retrying the step. If the error persists or if the retry attempt also fails, the system may then automatically retrieve a stored sample portion from a previous, stable point in the process. This stored sample portion serves as a backup, allowing the system to retry the process from a known “good” state without waiting for human intervention. By leveraging these stored samples, the system promotes continuity in the workflow, efficiently bypassing problematic steps or equipment that caused the initial error.

In an aspect, the predetermined storage duration may be managed by the MES and/or scheduler, one or both of which continuously, or periodically, monitor the status and progress of each sample. In this regard, the storage time and conditions for each sample portion may be logged into LIMS, thereby providing a comprehensive record that may be easily accessed and reviewed. In an aspect, if the predetermined storage period approaches its end, the system may evaluate the necessity of retaining the sample based on the current assay status and any pending validations. If the results have been confirmed and there are no pending issues and no additional value to be derived from the samples (e.g., the sample returns a negative result and no follow-up investigation is expected, requested, or pending), the system may decide to dispose of the sample portion, freeing up storage space for new samples.

In an aspect, the system may deploy a process enabling real-time error detection that allows the system to continuously or periodically monitor each step of the assay process, identifying any deviations from expected outcomes and triggering corrective actions. In an aspect, this real-time error detection mechanism may operate through a network of sensors strategically placed throughout the system. These sensors may continuously gather data on various parameters, such as temperature, pressure, volume, and timing, ensuring that aspects of the assay process adhere to the predefined protocols. For example, during a DNA extraction process, sensors may monitor the temperature of heating elements, the volume of reagents dispensed, and the duration of centrifugation steps. Any discrepancies from the expected values may be flagged by the system.

In an aspect, upon detecting an anomaly, the system's hierarchical architecture may analyze the data to determine the severity and potential impact of the error. Minor issues, such as slight deviations in reagent volumes, may be automatically corrected through real-time adjustments. However, more significant problems, such as complete failure of a critical piece of equipment, may trigger an alert within the system and prompt a series of predefined actions. These actions may include halting the current process, initiating alternative workflows, and notifying laboratory personnel.

In an aspect, the integration of real-time error detection with LIMS ensures comprehensive tracking and documentation of each identified issue. Errors and their corresponding corrective actions may be logged in LIMS, providing a detailed record that may be reviewed for quality control, auditing, and future optimization of the assay process. This logging capability may be important for maintaining transparency and traceability in high-throughput environments, where large volumes of samples are processed continuously.

When an anomaly or error is identified during any stage of the assay process, the system may employ advanced algorithms and decision-making protocols that may determine the best course of action. This process may involve evaluating one or more of the nature and severity of the errors, the stage at which it occurred, and the availability of stored sample portions that may be used for reprocessing. In an aspect, upon detecting an error, the system's hierarchical architecture (e.g., the MES) may assess the situation by, for instance, considering factors, including one or more of: the type of error (e.g., equipment malfunction, reagent issue, an issue detected with the sample, or procedural deviation), the criticality of the step during which the error occurred, the potential impact it may have on the overall assay results, and the availability of a previously stored portion of that sample to re-run. For example, an error in a critical step like DNA extraction may necessitate immediate reprocessing, whereas a minor deviation in a less critical step may be corrected in real-time without needing to revert to a previous stage.

In an aspect, the system may leverage data from LIMS to inform its decision-making process. LIMS may archive comprehensive records of all stored sample portions, including their unique identifiers, storage conditions and storage location, and the specific steps they have completed. This data enables the system to identify and retrieve the most appropriate stored sample for reprocessing. The decision-making algorithms may also consider the stability and quality of the stored samples, ensuring that only viable samples are used for reprocessing to maintain the integrity of the assay results.

In an aspect, if the system determines that reprocessing is necessary, it may autonomously retrieve the stored sample portion from the relevant storage unit and reinitiate the assay process from the appropriate step. In this regard, upon deciding to reprocess a sample, the system may first locate the stored portion in its integrated storage units (e.g., by accessing the comprehensive data logs in LIMS). The system accesses LIMS to pinpoint, e.g., the exact location, storage conditions, and status of the required sampled portion. In an aspect, the retrieval process may be fully automated, leveraging advanced robotic systems and conveyance mechanisms associated with the storage units. The robotic arms within a work cell may handle the samples so as to avoid contamination or degradation. Once the sample is retrieved, the system may prepare to initiate the processing from the specific step where the error-free sample portion was last correctly processed. In an aspect, detailed protocols stored within the system may outline the exact parameters and conditions required for each step. The system may revalidate the sample and the conditions of the reprocessing step, confirming that all necessary reagents, equipment, and settings are in place before proceeding.

The foregoing processes may ensure that samples are available for reprocessing, if needed. The system may also optimize the use of storage resources. Specifically, by maintaining samples only as long as necessary, the system may avoid unnecessary storage costs and potential degradation of biological materials over extended periods.

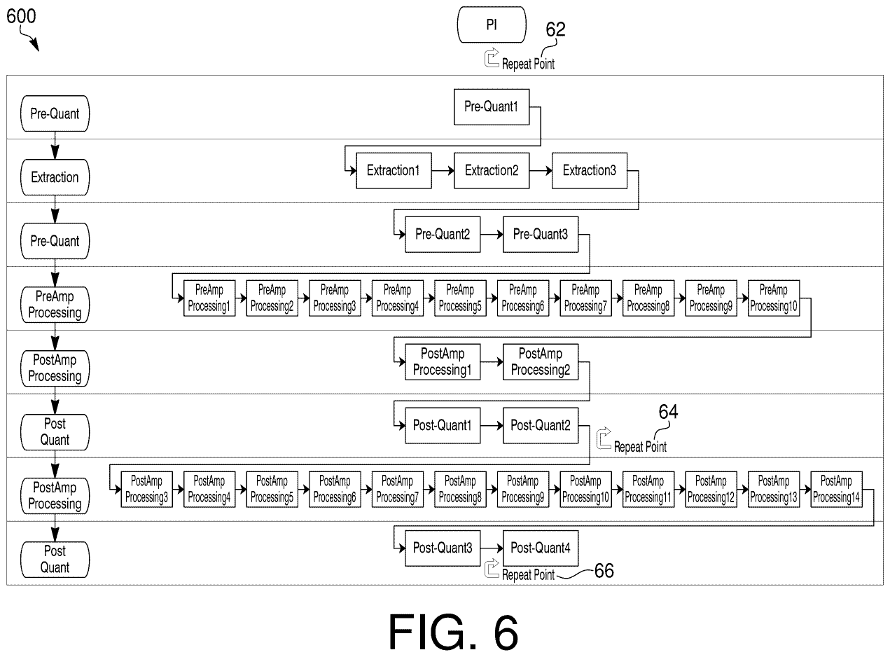

Referring now to FIG. 6, diagram 600 is presented that designates a position of repeat points in a multi-stage process for the automated handling and processing of biological samples, specifically focusing on tasks such as sample preparation, DNA extraction, pre-amplification processing, post-amplification processing, and sequencing preparation. Each stage is represented by distinct blocks and flow arrows that indicate the sequence of steps with the stage.

In FIG. 6, the repeat points 62, 64, 66 illustrated in diagram 600 represent specific points in the workflow where stored sample portions may be generated and retrieved from if an error is detected downstream or if there is a need for reprocessing. In an aspect, these repeat points 62, 64, 66 may be strategically placed at steps in the workflow where certain processes have been completed, where storage is possible because samples at that point in processing are stable enough for storage, or where it may be convenient to restart from if downstream issues occur. For instance, these repeat points 62, 64, 66 may be chosen based on the importance of the preceding step, the likelihood of errors occurring in subsequent steps, and/or the potential impact of those errors on the overall assay results. In an aspect, at each repeat point 62, 64, 66, a portion of the sample that has reached a stable state may be stored under controlled conditions. The storage units may be designed to maintain one or more specific conditions (e.g., temperature, humidity, etc.) suitable for preserving the integrity of the stored samples. If an error is detected in a downstream step or a repeat is otherwise requested, the system may automatically retrieve the stored sample portion from the relevant repeat point 62, 64, 66. This retrieval process may be fully automated, utilizing one or more robotic components and conveyance mechanisms (e.g., the centralized conveyance platform), to promote precision and avoid contamination. Once retrieved, the system may reinitiate the assay process from the repeat point 62, 64, 66, thus bypassing the need to start processing from the beginning. This may also save time when reprocessing is necessary, because a second portion of the sample may not need to start from the very first step of the process if there is an intermediate repeat point 62, 64, 66 present.

Diagram 600 illustrates three non-limiting, exemplary repeat points organized throughout the system workflow. The first repeat point 62 may be positioned prior to the initiation of the pre-quant workflow. In some aspects, this may coincide with the point in the workflow before anything has been added to the sample. As an example, the pre-quant workflow may follow a step of the workflow in which components of the sample are separated and isolated (e.g., in a whole blood sample, the plasma may be isolated; in a urine sample, the urine may be concentrated; in a liquid biopsy sample generally, the DNA or cfDNA may be isolated). A repeat point here may allow for reprocessing before the sample has been adulterated in any manner. In another aspect, the repeat point may coincide with a control addition step. A control addition step is a point in the workflow where control samples or reagents are introduced to ensure the integrity and accuracy of the assay process. A repeat point here may allow for reprocessing if any issues are detected with the control addition, such as improper reagent mixing, incorrect volumes or reagents and samples mixed, or contamination. A second repeat point 64 in diagram 600 is positioned after the post-quant processing and prior to post-amplification processing. In an aspect, at this point in the workflow, libraries that have been successfully prepared and normalized are stored. Because library preparation involves several precise tasks that are important for the quality of the final sequencing results, having a repeat point here may allow the system to handle errors or inconsistencies without having to redo all previous sequencing tasks. If errors occur in subsequent steps, such as during enrichment or sequencing preparation, the system may retrieve the stored libraries and restart the process from this point 64. A third repeat point 66 in diagram 600 is positioned after the preparation of sequencing libraries and before samples are pooled for sequencing. This repeat point 66 allows for the correction of errors that may occur during sequencing or to enable repeated sequencing of samples without needing to re-run the entire identical pool of samples.

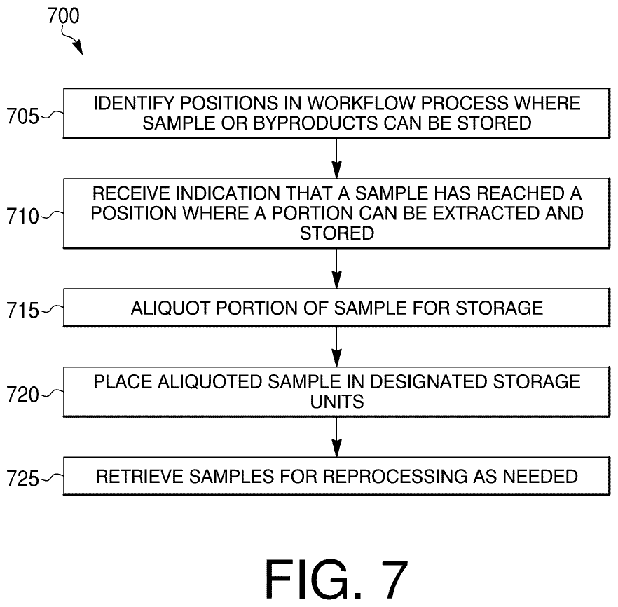

Referring now to FIG. 7, an exemplary flow diagram 700 is provided that delineates a decision-making process engaged in by system 100 associated with automatically storing and retrieving samples for reprocessing. At step 705, system 100 may identify the positions in the workflow process where sample portions or byproducts should be stored. These positions may be based on criteria such as achieving processing milestones, changes in sample characteristics, or when samples have reached a stable state with a sufficient shelf life. At step 710, system 100 may receive an indication or computationally determine that a sample, or set of samples, has reached a position in the workflow process where a portion of the sample may be extracted and stored. At step 715, system 100 may automatically aliquot a portion of the sample for storage. This process may be controlled to maintain sample integrity and prevent contamination. In some aspects, before aliquoting a portion of the sample for storage, a determination may be made regarding whether there is enough volume of sample to perform step 715, such that the primary sample to be processed and the secondary sample to be stored for potential reprocessing both would have sufficient volume to process the samples. If an insufficient volume of sample is available, then method 700 may skip steps 715 through 725 and may not store a secondary portion of the sample for potential reprocessing. In some aspects, if aliquoting is performed, the sample may be divided into two approximately equal portions, or a predetermined amount of sample may be aliquoted from the sample to form the secondary sample for storage.

At step 720, system 100 may place aliquoted samples in designated storage units that may be equipped to maintain the necessary environmental conditions (e.g., temperature, humidity, etc.) based on the type of sample to be stored. In some aspects, during performance of either step 715 or step 720, the aliquoted sample may be portioned into a container that is labeled to indicate which sample the aliquoted sample is matched with, and thus to identify the sample correctly if the matching sample needs to be reprocessed. In some aspects, labeling or aliquoting into a previously labeled container may occur during step 715, and step 720 may include scanning the labeled container of the aliquoted sample as it is stored in the designated storage unit and recording the location of the aliquoted sample in the storage unit to LIMS. In some aspects, system 100 may utilize storage units with multi-temperature compartments if needed to accommodate different storage requirements.

At step 725, system 100 may be configured to retrieve the stored samples as needed. For instance, if an error is detected and/or reprocessing is required, system 100 may automatically retrieve the stored sample portion from the relevant storage unit that is paired with the sample that needs to be reprocessed.

### Crash Recovery

Conventional laboratory systems often struggle with effectively managing expected shutdowns, such as planned power outages or system shutdowns, e.g., due to maintenance in the area or extreme events, such as hurricanes or wildfires. Moreover, conventional laboratory systems also struggle with effectively managing unexpected shutdowns, such as those caused by power outages or IT failures. Such planned or unplanned shutdowns may lead to significant risks, including the loss of valuable samples or data. These systems generally lack advanced crash recovery mechanisms, resulting in scenarios where samples may be left in an unstable state, potentially compromising their integrity and rendering them unusable. This gap in conventional systems highlights the critical need for a robust recovery protocol that promotes continuity and protection of samples or resources during unforeseen disruptions.

The system may leverage a unique and sophisticated crash recovery mechanism designed to maintain sample integrity, even in the face of unexpected shutdowns. This mechanism is configured to continue processing samples until they reach predefined “safe points” before the system fully shuts down. These safe points may be selected logical and physical locations within the workflow where samples may be safely stored for extended periods without risk of degradation for prolonged periods of time. For instance, samples may be moved to cold storage (e.g., within a storage unit within a buffer or activity work cell), where they may remain stable for up to a predetermined period of time, e.g., up to 12 hours, up to 18 hours, up to 24 hours, up to 48 hours, up to 72 hours, or longer, depending on the sample type and storage conditions.

In an aspect, the system's shutdown protocol may be designed to handle both anticipated and unanticipated events. For instance, with respect to the former, regularly planned maintenance activities (e.g., software and/or hardware updates, routine instrument calibrations, etc.) may necessitate a controlled shutdown. As another example, pre-announced power grid maintenance or other types of electrical work may require the system to prepare for a temporary loss of power. In yet another example, severe wildfire or weather forecasts that may threaten power supplies and/or other types of infrastructure (e.g., thunderstorms, hurricanes, ice storms, etc.) may trigger a planned shutdown. With respect to the latter, one example of an unanticipated event may correspond to an unexpected power outage (e.g., resultant from faults in the electrical grid, equipment failures, sudden natural disasters, etc.). Another example may relate to unforeseen breakdowns of critical laboratory equipment, such as robotic arms, centrifuges, temperature control systems, etc., which may disrupt sample processing.

When a shutdown is initiated, either manually or automatically in response to a detected issue, the system ensures that all in-process samples are processed to the nearest safe point before halting operations. This protocol is important for maintaining the stability and viability of samples, as it prevents them from being abandoned mid-process, which may lead to contamination, degradation, or data loss. In an aspect, the system may be designed to include multiple safe points throughout the workflow, so that no sample is ever more than a predefined working period of time (e.g., approximately one hour, approximately two hours, approximately three hours, approximately four hours, approximately five hours, approximately six hours, approximately seven hours, approximately eight hours, etc.) away from a secure stopping point, thereby inhibiting the risk of loss during a shutdown. In some aspects, a sample may be approximately one hour to approximately eight hours away from a safe point, may be approximately two hours to approximately six hours from a safe point, may be approximately four hours to approximately eight hours, etc. In an aspect, the system's crash recovery mechanism may account for the varying stages of sample processing, as some samples may be closer to a safe point than others. More particularly, depending on where a sample is in the process when a shutdown occurs (both physically and logically within the workflow), it may require more or fewer steps to reach the next safe point. For instance, a sample that is nearing the end of a particular step, such as the final phase of DNA extraction, may only need one more action (e.g., such as transferring to a cold storage unit) to reach a safe point. Conversely, samples in earlier portions of a complex stage may require several additional steps before they can be safely halted. This variation may require the system to dynamically assess the position of each sample and prioritize tasks so that all samples can be stabilized as quickly as possible.

In an aspect, the positioning of stopping points within the system may not be randomly placed, but rather, may be strategically and logically positioned within the workflow, e.g., based on the specific type of sample being processed and/or the nature of the assay being conducted. In an aspect, the system may be designed to recognize the unique requirements of different sample types, such as blood, urine, tissue, or DNA (including fragments), and the specific conditions each requires to remain stable during processing, or whether it may not be possible for a sample to be stable during a certain processing step. For example, biological samples like RNA, which are highly sensitive to degradation, may necessitate stopping points that include ultra-low temperature storage to preserve their integrity. Similarly, the type of assay being performed (e.g., whether it be DNA sequencing, PCR, protein analysis, etc.), may at least in part dictate the optimal stopping points within the workflow. For instance, during a multi-step assay like DNA extraction, logical stopping points may be placed after the initial lysis step, where the DNA is sufficiently stabilized in a buffer, or after the purification step, where the extracted DNA may be stored at cold temperatures to prevent degradation. The stopping points may ensure that the sample can be safely paused at key stages without substantially compromising the quality or accuracy of the subsequent assay results.

Moreover, the system may dynamically adjust the placement of these stopping points based on real-time data and workflow progression. For instance, if the system detects that a sample batch is behind schedule or if there's a risk of a system shutdown, it may designate an earlier stopping point to ensure that the samples reach a safe state more quickly. This flexibility may allow the system to handle a wide variety of assays and sample types, ensuring that each sample is preserved in the best possible condition, ready to resume processing with minimal disruption once the system is back online. In some aspects, a user may indicate an amount of time available until a shutdown needs to occur. If sufficient time is provided, the system may continue processing the samples until ideal, primary, shutdown states are reached by each sample. However, if a shorter amount of time until shutdown is indicated, then the system may continue processing until shorter secondary shutdown states are reached by each sample, if needed. This secondary state is a more immediate, interim point where samples can be safely paused without completing all the steps required for the primary state, but still ensuring minimal risk to sample integrity. Alternatively, in some configurations, the system may be designed with only one set of predefined shutdown states, rather than having both primary and secondary shutdown states. This approach simplifies the shutdown protocol, focusing on a single, well-defined set of conditions under which all samples can be safely stored until power and/or system functionality is restored.

Additionally, the safe storage points may be designed to accommodate the unique needs of various types of samples and assay processes. In an aspect, these storage points may differ in their capabilities, particularly in terms of temperature control and the duration for which samples can be safely stored. The diversity in storage conditions may allow the system to flexibly manage samples, ensuring their integrity and usability across a wide range of scenarios. One type of safe storage may be an ambient temperature storage point. These storage points may maintain samples at room temperature (e.g., approximately 20° C. to 25° C.) and may typically be used for short-term storage of samples that are stable at room temperature. These may include samples that have been treated with preservatives or are inherently stable over short periods. Another type of safe storage point may be a refrigerated storage point, which may be configured to maintain a cool environment (e.g., approximately 4° C.) and is ideal for preserving biological samples that require a stable, cold environment to prevent degradation over moderate timeframes. Yet another type of safe storage point may be a low-temperature storage point (e.g., maintained at approximately −20° C., approximately −40° C., or approximately −80° C., etc.), which may be ideal for preserving highly sensitive samples, e.g., such as RNA or certain enzymes, which are prone to rapid degradation at higher temperatures. The appropriate type of safe storage may depend, at least in part, on where in the process a sample is to be stored, and the appropriate storage type may be included at that location in the system.

In an aspect, to be able to perform the processes described above when facing unexpected situations (e.g., a power outage) that starts with little or no warning, the system may be connected to one or more emergency energy sources (such as batteries, backup generators, etc.) to effectively manage sample processing and storage. The emergency energy sources may provide the necessary backup power to allow the system to execute essential steps that enable each sample to reach a predefined safe point. Additionally, the role of the emergency energy sources may not only be to power the system's immediate processing needs to reach these safe points, but also to maintain the refrigeration units at optimal temperatures for extended periods, if necessary. This continuous supply of power may preserve the integrity of sensitive biological samples, such as RNA or enzymes, which may require specific storage conditions to remain stable.

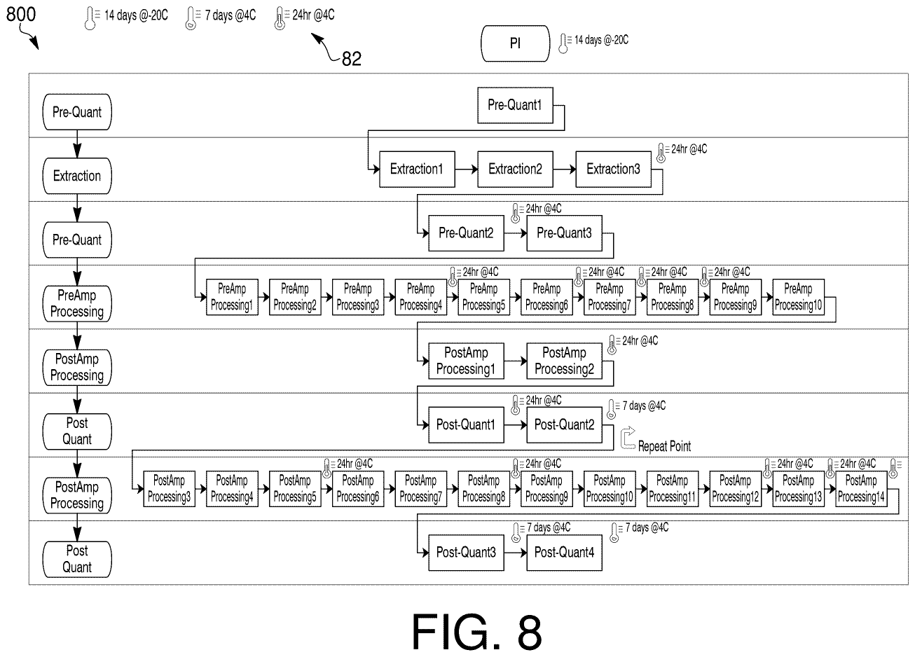

Referring now to FIG. 8, diagram 800 is presented that represents an exemplary workflow for the automated handling and processing of biological samples that contains various types of safe storage points 82 that are placed throughout the workflow to promote sample integrity and stability during both planned and unplanned system shutdowns. To illustrate the concepts described above in this section, a non-limiting example of a system shutdown process is described. In this example, the system may be performing a workflow that involves extracting cell-free DNA (cfDNA) from whole blood samples. During the processing of samples at the extraction normalization step, the system may receive a command to initiate a factory shutdown. This may be due to a variety of reasons, such as an impending power outage, scheduled maintenance, or an unforeseen equipment failure. In an aspect, upon receiving the shutdown command, the system's control software may assess the current state of all in-process samples and may identify the nearest logical stopping point for each sample batch. In the current case, the system may determine that the samples having just completed a plasma isolation step may continue processing through the new few steps (and/or work cells) through the completion of the extraction step before being placed into safe storage. Once the work on the extraction work cell has completed, the system may recognize that it has reached a predefined safe storage point. The DNA samples may then be in a stable, processed state, with all necessary preparatory steps completed up to this point. This safe storage point may be chosen because, at this point in the workflow, the DNA is adequately stabilized in a buffer or reagent mix that can withstand longer storage durations without risk of degradation. In some aspects, the system may log this transition into a temporary storage into LIMS, including details of the event such as the time, date, and conditions under which the samples were stored.

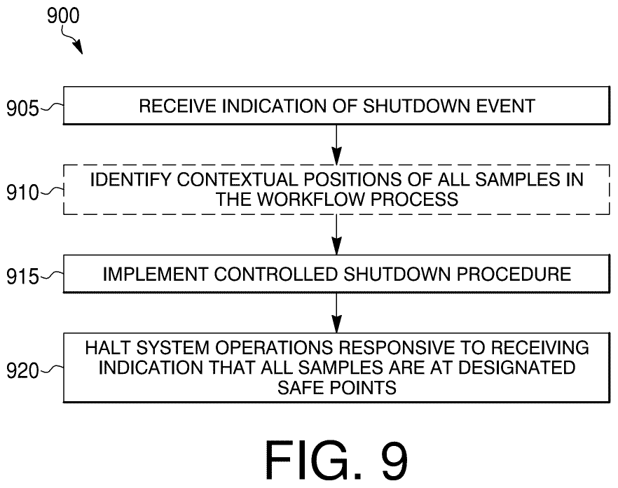

Referring now to FIG. 9, an exemplary flow diagram 900 is provided that delineates a decision-making process engaged in by system 100. At step 905, system 100 may receive an indication of a shutdown event. The shutdown event may be a planned shutdown (e.g., resultant from maintenance, software updates, etc.) or unplanned (e.g., power failure, natural disaster, etc.). At step 910, in an aspect, system 100 may evaluate the progress and status of all samples currently being processed. This may involve identifying the specific task, step, and/or stage each sample is associated with in the workflow process and the proximity of each sample to the next predefined safe point. In some aspects, step 910 may be optional. More particularly, rather than evaluating the contextual position of each sample, system 100 may simply go to step 915 and implement the controlled shutdown procedure. In this regard, the controlled shutdown procedure may involve processing samples to their next respective safe points, regardless of their contextual position. System 100 may facilitate this by transmitting the shutdown procedure instructions to the necessary work cells and/or components of the conveyor platform. At step 920, system 100 may halt system operations responsive to determining and/or receiving an indication that all samples are at a safe stopping point. In some situations, system 100 may halt system operations before all samples are at their respective safe stopping points. For example, when the system 100 identifies that a shutdown is imminent and a specific threshold number or percentage of samples have reached their safe stopping point, system 100 may choose to preserve the rest of the samples that have made it to the safe stopping point rather than risking system integrity by waiting for the final samples to get to their safe stopping points.

### Optimized Dwell Time

In an aspect, the system may incorporate a dynamic mechanism for optimizing dwell time to ensure an efficient processing flow throughout the assay workflow. Dwell time refers to the period of time that samples or materials spend at a particular stage in the workflow before moving on to the next step. In traditional systems, dwell times can often be static and predetermined, leading to inefficiencies such as unnecessary waiting periods or processing delays, especially when the system encounters variability in sample loads or unexpected bottlenecks.

In the disclosed system, dwell times may be dynamically adjusted based on real-time data and workflow demands. In one aspect, this dynamic adjustment may be managed by the MES layer, which may continuously or periodically monitor the status of samples, work cells, and resources, making real-time decisions on how long a sample should remain at each stage before proceeding. By fine-tuning these dwell times, the system promotes efficient processing of samples, inhibiting idle time and preventing work cells from becoming bottlenecks in the workflow. For example, if a particular work cell is experiencing a delay due to resource replenishment or maintenance, the MES may adjust the dwell time of samples in preceding stages, holding them at earlier points in the workflow until the work cell is ready to process them. Conversely, if a work cell is operating above capacity, the system may reduce dwell times in earlier stages to feed more samples into the work cell, thereby maximizing its utilization.

To implement dwell time adjustment, the MES may communicate with the relevant scheduler layer(s). For instance, suppose the MES identifies that a step is completed faster than anticipated and the next step is ready to begin. The MES may send a command to the relevant scheduler to decrease the dwell time for samples in the intermediate buffer work cell, allowing them to be promptly moved to the activity work cell. In an aspect, the communication between the MES and the scheduler layer may be continuous or regular and dynamic, allowing for real-time adjustments to be made as conditions change throughout the processing line. By leveraging the scheduler's ability to precisely control equipment and resource allocation within a work cell, the MES may fine-tune the processing environment to accommodate various operational scenarios.

As a non-limiting example of the foregoing, an example of optimizing dwell time during sample processing may be seen during the DNA extraction and purification phase. For instance, a batch of samples may be undergoing a multi-step process that includes DNA extraction, washing, elution, and subsequent quantification. Each of these steps requires precise timing to promote sample integrity and the efficiency of the workflow. During DNA extraction, the DNA may be bound to a solid-phase support, followed by a series of washing steps to remove contaminants. In a conventional system, samples may remain in the washing step for a fixed duration, regardless of the actual conditions or the status of downstream processes.

However, in aspects of the optimized system described herein, the MES layer may dynamically adjust the dwell time based on real-time data. For instance, if the MES detects that the next processing step, such as DNA elution, is momentarily delayed due to a bottleneck in the conveyance platform or a temporary unavailability of reagents, it may extend the dwell time in the washing step. This extension may prevent the samples from being prematurely moved to the next step where they would have to wait, potentially degrading or becoming contaminated. Conversely, if the downstream processes are ready sooner than expected, the MES may shorten the dwell time in the washing step, allowing the samples to move on more quickly, thereby reducing overall processing time.

### Dynamic Identification of Storage Points to Accommodate Processing Decisions

The dynamic identification of storage points may promote the system's ability to maintain workflow continuity and safeguard sample integrity, even as the top software layer processes and analyzes data to make informed decisions. Given the complexity and volume of data the MES handles—such as real-time status updates from work cells, inventory levels, and error reports—there can be delays in issuing new instructions for the next steps in the processing workflow. To ensure that these delays do not disrupt the overall process or compromise the samples, the system may incorporate strategically designed “stopping points” where samples can safely pause.

These stopping points may be predetermined locations within the workflow where samples can be temporarily stored without risk to their quality or stability. Each stopping point may be typically associated with a storage environment that is configured to maintain conditions conducive to preserving the samples' integrity, such as cool storage or stable chemical environments. For instance, after completing a particular process step, a sample may be placed in a refrigerated storage compartment that inhibits degradation while the MES determines the next processing instructions. This ensures that samples are not left in unstable or vulnerable conditions during periods of inactivity.

In an aspect, the design of processes within the system may take into account these stopping points, ensuring that at the end of each step, the sample may be safely held in a stable environment if immediate continuation of processing is not possible. This approach may protect the samples from potential harm and may also allow the system to manage high throughput effectively. By having these dynamic storage points, the system may continue to operate efficiently, even when complex data processing and decision-making are required by the MES.

### Sample Processing Overview

The system described herein may be fully automated and designed to handle biological samples from receipt to the generation of analysis results. The system may incorporate various work cells, a conveyance platform, and a hierarchical software structure that coordinates and manages the entire process.

In an aspect, when samples arrive at the laboratory, they may first be registered into LIMS. Each sample may be given a unique identifier (e.g., a barcode, QR code, or RFID tag) that will be used to track the sample batch throughout the entire process. Samples may then be transferred to a central receiving station, where they may be prepared for entry into the automated system. In an aspect, samples may then be transported through a series of work cells, which will collectively perform various steps to complete various stages of the processing workflow. Each of these work cells may be equipped with the appropriate instruments and materials needed for the sample processing activities that occur at that particular stage of the workflow. In an aspect, sample and material transport may be handled by the MES via control of a conveyance platform.

As a non-limiting example of an exemplary workflow process, samples may originally be transported to an initial preparatory work cell where the original sample is separated to isolate certain components to be used during the workflow. As an example, in aspects where the original sample is provided as a whole blood sample, the sample may be subjected to centrifugation and selective aspiration to isolate the plasma from the sample. As another example, in aspects where the original sample is provided as urine, the sample may be filtered and concentrated. Samples may then be transported to one or more extraction work cells, where specific components, such as DNA, RNA, or proteins, are further isolated. The extraction process may involve multiple steps, including digestion, lysis, and separation, conducted within the extraction work cell. In an aspect, one or more robotic components (e.g., robotic arms) may aid in the performance of these tasks (e.g., via performing the task itself or by ferrying samples between the relevant instruments in the work cell). The extracted material from each sample is either directly moved to the next processing stage or is temporarily stored in a storage location if further processing is delayed. After extraction, the samples may be conveyed to quantification work cells, where the concentration of the extracted components is measured. Automated instruments within these work cells (e.g., spectrophotometers or fluorometers, etc.) may provide quantification data. Based on these measurements, samples may then be normalized to a standard concentration to promote uniformity for downstream processing. In an aspect, the scheduler layer software may coordinate these activities, adjusting dwell times as necessary to prevent bottlenecks.

Samples may then be moved to a pre-amplification work cell, where reagents necessary for amplification, such as primers and enzymes, are added. This work cell may be equipped with robotic pipetting systems that add reagents based on the workflow process specified by the MES. Once reagents are added, the samples may be mixed thoroughly and prepared for thermal cycling. The samples may then be transported to a thermal cycling work cell, where they undergo Polymerase Chain Reaction (PCR) or other amplification processes. The scheduler for the thermal cycling work cell may monitor the progress of amplification, ensuring that each sample is processed according to its unique protocol requirements.

Upon completion of amplification, the samples may be conveyed to the post-amplification work cells, where they may undergo further processing steps such as purification, cleanup, or preparation for sequencing. In an aspect, robotic components may perform these tasks, transferring samples to and from different instruments within the work cell as required. In the sequencing preparation work cells, the samples may be prepared for sequencing by, for example, adding sequencing adapters, performing size selection, or performing quality checks. Similar to the foregoing, robotic components may handle these operations to promote consistency and precision. Samples may then be transferred to sequencing work cells, which include advanced sequencing instruments that are configured to perform high-throughput sequencing, thereby generating large volumes of data. As sequencing progresses, raw data may be collected and transferred to the data analysis work cells. Here, computational systems may process the data using bioinformatics tools to generate interpretable results. This may involve aligning sequences to reference genomes, identifying variants, or quantifying expression levels.

In some aspects, the system may employ validation safeguards where the generated data is validated against predefined criteria to assess accuracy and reliability. Any discrepancies or anomalies may be flagged for review. If necessary, the system may use previously stored samples to reprocess specific steps or confirm results. In an aspect, once the data has passed quality control, final results may be generated and stored in LIMS or another storage location. These results may then be accessed by researchers, clinicians, patients, or other stakeholders. The system may also log all process data, providing a comprehensive audit trail for future reference. In an aspect, after processing, any remaining samples may either be stored for potential future use or disposed of according to conventional laboratory protocols. In some aspects, after results associated with a sample are provided to a recipient (e.g., a clinician or a patient from whom the sample was obtained), the sample may be discarded.

In an aspect, between processing steps, samples may be placed in buffer work cells equipped with local storage compartments. These compartments may be designed to maintain samples at specific conditions (e.g., temperature and humidity) to inhibit sample degradation and maintain stability. This intermediate storage may allow system 100 to manage workflow transitions and promote readiness of samples for the next steps without unnecessary delays.

In some aspects, the autonomous laboratory system may further include a quality control (QC) and contamination detection subsystem that may be configured to autonomously monitor, evaluate, and respond to deviations in sample integrity, assay performance, or environmental conditions occurring during execution of the workflow process. In an aspect, the QC and contamination detection subsystem may continuously or periodically analyze data published to LIMS, including, e.g., instrument health indications generated by the one or more third software layers, reagent usage patterns, environmental sensor readings within a work cell, and sample-level processing metrics recorded by the one or more second software layers. For example, the QC and contamination detection subsystem may detect a sudden increase in particulate counts from environmental sensors within a work cell while also observing abnormal reagent consumption and elevated instrument temperature readings published to LIMS, collectively indicating a potential contamination event. In response, the subsystem may flag the anomaly and notify the MES to initiate an appropriate corrective action sequence.

Upon detecting a contamination event, anomalous assay behavior, or a deviation from expected performance thresholds, the QC and contamination detection subsystem may transmit an indication to the MES, which may automatically initiate a corrective response sequence that leverages the system's existing safe-point and reprocessing architecture. For instance, responsive to detecting a contamination event in an activity work cell, the MES may determine the most recent safe-point associated with the affected sample or batch, quarantine the impacted work cell or instruments, reroute samples to alternate work cells, and instruct the relevant scheduler to reprocess the samples from the designated safe-point. In some aspects, the subsystem may additionally trigger automated decontamination procedures, adjust workflow routing to avoid temporarily offline equipment, or initiate retesting protocols to validate sample integrity.

In general, any process discussed in this disclosure that is understood to be computer-implementable may be performed by one or more processors of a computer system, such as system environment 100, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer server. A processor may be a central processing unit (CPU), a graphics processing unit (GPU), or any suitable types of processing unit.

A computer system, such as system environment 110, may include one or more computing devices. If the one or more processors of the computer system are implemented as a plurality of processors, the plurality of processors may be included in a single computing device or distributed among a plurality of computing devices. If a system environment comprises a plurality of computing devices, the memory of the computer system may include the respective memory of each computing device of the plurality of computing devices.

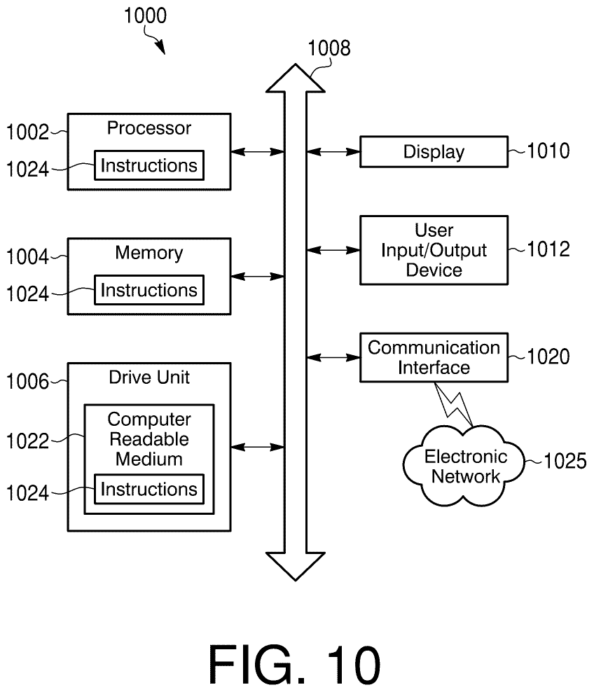

FIG. 10 is a simplified functional block diagram of a computer system 1000 that may be configured as a computing device for executing the processes described herein, according to exemplary embodiments of the present disclosure. FIG. 10 is a simplified functional block diagram of a computer that may be configured according to exemplary embodiments of the present disclosure. In various embodiments, any of the systems herein may be an assembly of hardware including, for example, a data communication interface 1020 for packet data communication. The platform also may include a central processing unit (“CPU”) 1002, in the form of one or more processors, for executing program instructions. The platform may include an internal communication bus 1008, and a storage unit 1006 (such as ROM, HDD, SDD, etc.) that may store data on a computer readable medium 1022, although the system 1000 may receive programming and data via network communications via electronic network 1025 (e.g., voice, video, audio, images, or any other data over the electronic network 1025). The system 1000 may also have a memory 1004 (such as RAM) storing instructions 1024 for executing techniques presented herein, although the instructions 1024 may be stored temporarily or permanently within other modules of system 1000 (e.g., processor 1002 and/or computer readable medium 1022). The system 1000 also may include input and output ports 1012 and/or a display 1010 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. Relative terms, such as “about,” “approximately,” “substantially,” and “generally,” are used to indicate a possible variation of ±10% of a stated or understood value. In addition, the term “between” used in describing ranges of values is intended to include the minimum and maximum values described herein. The use of the term “or” in the claims and specification is used to mean “and/or” unless explicitly indicated to refer to alternatives only if the alternatives are mutually exclusive, although the disclosure supports a definition that refers to only alternatives and “and/or.” As used herein “another” may mean at least a second or more.

As used herein, the term “user” generally encompasses any person or entity, such as a researcher and/or a care provider (e.g., a doctor, etc.), that may desire information, resolution of an issue, or engage in any other type of interaction with a provider of the systems and methods described herein (e.g., via an application interface resident on their electronic device, etc.). The term “electronic application” or “application” may be used interchangeably with other terms like “program,” or the like, and generally encompasses software that is configured to interact with, modify, override, supplement, or operate in conjunction with other software.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

1. An automated sample processing system, the system comprising:
one or more processors;
one or more computer readable media storing instructions executable by the one or more processors to:
identify that a sample in a laboratory workflow controlled by the system is in a buffer work cell;
receive operational context data associated with the laboratory workflow indicating an event affecting a subsequent processing step for the sample in the laboratory workflow; and
dynamically adjust a dwell time for the sample in the buffer work cell based on the received operational context data.

2.-8. (canceled)

9. An automated sample processing system, the system comprising:
one or more processors;
one or more computer readable media storing instructions executable by the one or more processors to:
receive an indication that an amount of a material at a local storage compartment associated with a laboratory workflow has fallen below a predetermined threshold;
determine a status of one or more samples being processed in accordance with the laboratory workflow and, depending on the status, a projected movement of the one or more samples on a conveyance platform of the system; and
initiate a replenishment protocol responsive to receiving the indication to increase the amount of the material above the predetermined threshold and based on the projected movement of the one or more samples on the conveyance platform.

10.-19. (canceled)

20. An automated sample processing system, the system comprising:
one or more processors;
one or more computer readable media storing instructions executable by the one or more processors to:
receive an indication of a shutdown event for a laboratory workflow controlled by the system;
implement a shutdown procedure to address the shutdown event, wherein the shutdown procedure causes one or more samples involved in the laboratory workflow to continue processing to a predefined safe stopping point; and
halt at least a subset of the operations of the system at a conclusion of the shutdown procedure.

21.-36. (canceled)

37. An automated sample processing system, the system comprising:
one or more processors;
one or more computer readable media storing instructions executable by the one or more processors to:
identify a position of a sample in a laboratory workflow controlled by the system;
determine that the position of the sample in the laboratory workflow corresponds to an automatic storage position;
cause a portion of the sample to be aliquoted to create a matched aliquoted sample; and
store, responsive to the determining, the matched aliquoted sample in a storage location.

38. The system of claim 37, wherein the instructions executable by the processor to store the matched aliquoted sample comprise instructions executable by the processor to store the matched aliquoted sample for a predetermined period of time.

39. The system of claim 38, wherein the sample is a biological sample, wherein the laboratory workflow is an analytical workflow, and wherein the predetermined period of time corresponds to a time it takes for a patient to receive a test result of the analytical workflow.

40. The system of claim 39, wherein the analytical workflow comprises a cancer determination workflow, and the test result indicates presence or a prediction of presence of: cancer, no cancer, or a type of cancer.

41. The system of claim 37, wherein the instructions executable by the processor to cause the component to aliquot the portion of the sample comprise instructions executable by the processor to:
determine there is enough volume of sample such that a remaining amount of the sample after being aliquoted and the aliquoted sample would both have sufficient volume to complete a subsequent processing step; and
cause the component to aliquot the portion from the sample such that the remaining portion of the sample is at least as large as the sufficient volume.

42. The system of claim 37, wherein the component is a robotic arm or automated pipettor.

43. The system of claim 37, wherein a temperature and type of the storage location is dependent, at least in part, on a characteristic of the sample.

44. The system of claim 37, wherein the instructions are further executable by the one or more processors to:
receive a directive to reprocess the sample;
retrieve the aliquoted sample from the storage location in response to the directive; and
transfer the aliquoted sample to a conveyance platform of the automated sample processing system for the aliquoted sample to be processed according to a reprocessing procedure.

45. The system of claim 44, wherein the directive is generated by the system in response to detecting a deviation in the laboratory workflow.

46. The system of claim 44, wherein the directive is generated by the system in response to detecting a deviation in the laboratory workflow that is greater than a predetermined threshold deviation.

47. The system of claim 44, wherein the reprocessing procedure is fully automated.

48. The system of claim 37, wherein the sample is a biological sample.

49. The system of claim 37, wherein the laboratory workflow is a genetic analysis workflow, a disease detection or classification workflow, a viral detection workflow, or a cancer detection or classification workflow.

50. The system of claim 37, wherein the system comprises a hierarchical software architecture comprising a plurality of software layers that are collectively configured to automate the laboratory workflow, wherein each of the plurality of software layers is configured to be ignorant to a functionality of another of the plurality of software layers, wherein the plurality of software layers comprise:
a first software layer of the plurality of software layers configured to guide the sample between one or more work cells during progression of the sample through the laboratory workflow, to identify the position of the sample in the laboratory workflow, and to determine that the position of the sample corresponds to the automatic storage position;
one or more second software layers of the plurality of software layers, wherein each of the one or more second software layers are configured to manage execution of one or more steps associated with the laboratory workflow process in the one or more work cells, to cause the instrument to create and store the aliquoted sample; and
one or more third software layers of the plurality of software layers, wherein each of the one or more third software layers are configured to manage execution of tasks, performed by an instrument of a plurality of instruments contained in the one or more work cells, in furtherance of the one or more steps.

51. The system of claim 50, wherein each of the one or more second software layers are associated with one of the one or more work cells.

52. The system of claim 50, wherein each of the one or more third software layers are associated with an instrument of the plurality of instruments contained in the one or more work cells.

53. An automated sample processing system, the system comprising:
one or more processors;
one or more computer readable media storing instructions executable by the one or more processors to:
identify a position of a sample in a laboratory workflow controlled by the system;
identify a status of an activity work cell in a laboratory workflow that is downstream of the position of the sample in the laboratory workflow;
determine that the status of the activity work cell with respect to the position of the sample requires placement of the sample in a buffer work cell; and
transport the sample to the buffer work cell.

54.-62. (canceled)
